(19) United States
(12) Patent Application Publication
(10) Pub. No.: US 2024/0174636 A1
(43) Pub. Date: May 30, 2024

(54) NOVEL INDOLEAMINE 2,3-DIOXYGENASE INHIBITORS, PROCESSES FOR THE PREPARATION THEREOF AND PHARMACEUTICAL COMPOSITIONS COMPRISING THE SAME

(71) Applicant: YUHAN CORPORATION, Seoul (KR)

(72) Inventors: Gyu-Jin LEE, Yongin-si, Gyeonggi-do (KR); Eui-Chul LEE, Suwon-si, Gyeonggi-do (KR); Sol PARK, Suwon-si, Gyeonggi-do (KR); Dong-Hoon LEE, Suwon-si, Gyeonggi-do (KR); Su-Hwa KIM, Yongin-si, Gyeonggi-do (KR); Jong-Suk PARK, Anyang-si, Gyeonggi-do (KR); Ho-Woong KANG, Daejeon (KR); Hyun-Sik YUN, Yongin-si, Gyeonggi-do (KR)

(73) Assignee: YUHAN CORPORATION, Seoul (KR)

(21) Appl. No.: 18/551,894

(22) PCT Filed: Mar. 22, 2022
(86) PCT No.: PCT/KR2022/003945

§ 371 Date: Sep. 22, 2023

(30) Foreign Application Priority Data
Mar. 24, 2021 (KR) 10-2021-0037824

(51) Int. Cl. C07D 401/12; A61K 31/4709; A61K 31/517; C07D 215/18; C07D 405/12; C07D 413/12; C07D 417/12; C07D 498/04; C07D 513/04

(52) U.S. Cl. CPC C07D 401/12; A61K 31/4709; A61K 31/517; C07D 215/18; C07D 405/12; C07D 413/12; C07D 417/12; C07D 498/04; C07D 513/04

(57) ABSTRACT

Provided are a compound having a cyclohexyl-(alkyl or cycloalkyl-substituted)ethylene-amino-heteroaryl moiety or pharmaceutically acceptable salt thereof, a process for the preparation thereof, a pharmaceutical composition comprising the same and a use thereof, wherein the compound or pharmaceutically acceptable salt thereof not only has excellent inhibitory activity against indoleamine 2,3-dioxygenase (IDO) but also exhibits remarkably high in vivo exposure upon oral administration and therefore the compound or pharmaceutically acceptable salt thereof can be usefully applied for preventing or treating various diseases associated with IDO.

### TECHNICAL FIELD

The present invention relates to a novel compound having an inhibitory activity against indoleamine 2,3-dioxygenase (IDO), i.e., derivatives having a cyclohexyl-(alkyl or cycloalkyl-substituted)ethylene-amino-heteroaryl moiety or pharmaceutically acceptable salt thereof, a process for the preparation thereof, a pharmaceutical composition comprising the same and a use thereof.

### BACKGROUND ART

Rapid proliferation of cancer is originated from the avoidance of attack by the immune system. It has been reported that tryptophan plays an important role for cancer immune evasion (Opitz, C. A. et al. Nature 478, 197-203 (2011)). In particular, the complex interaction between cancer cells and immune cells is one of the critical factors in determining the survival and death of early cancer cells. The pathways that cancer cells use to evade the attack of immune cells are related to the immune-suppressive pathway. Recently, the degradation process of tryptophan by indoleamine 2,3-dioxygenase (IDO) and the role of the resulting kynurenine are attracting attention.

Tryptophan is an essential amino acid for proliferation and survival of cells. Indoleamine 2,3-dioxygenase (conventionally, referred to as ‘IDO-1’) is an intracellular heme-containing enzyme that catalyzes the first and rate-limiting step of tryptophan degradation to N-formyl-kynurenine. IDO acts on the metabolism of L-tryptophan to degradate it into N-formyl-kynurenine, which is then metabolized by various steps to produce nicotinamide adenine dinucleotide (NAD+). Tryptophan catabolites produced from N-formyl-kynurenine, such as kynurenine, are known to be cytotoxic to T-cells. Therefore, IDO depletes tryptophan and produces kynurenine, thereby inhibiting the activity of immune cells, including T-cells, through various mechanisms (Mellor, A. L. & Munn, D. H. Nature Rev. Immunol. 8, 74-80 (2008), Fallarino, F., Gizzi, S., Mosci, P., Gronmann, U. & Puccetti, P. Curr. Drug Metab. 8, 209-216 (2007)). IDO is also distributed in dendritic cells and regulatory B cells (as well as cancer cells) and acts on these cells to suppress the ability of the immune system to recognize and attack cancer cells. Therefore, overexpression of IDO may lead to increased resistance in the tumor microenvironment, which results in growing cancer tissues.

It has been reported that the up-regulation of IDO leads to a poor prognosis in cancer patients (Uyttenhove, C. et al. Nature Med. 9, 1269-1274 (2003)). From the test using IDO gene knockout mice, it has been confirmed that IDO plays a key role in immune tolerance and inflammatory carcinogenesis (Muller, A. J., Mandik-Nayak, L. & Prendergast, G. C. Immunotherapy 2, 293-297 (2010) Muller, A. J. et al. Proc. Natl Acad. Sci. USA 105, 17073-17078 (2008)). Especially, it has been reported that the use of an IDO inhibitor as a supplemental treating agent improves the effects of immunochemotherapy, radiotherapy, and anticancer vaccines (Muller, A. J., DuHadaway, J. B., Donover, P. S., Sutanto-Ward, E. & Prendergast, G. C. Nature Med. 11, 312-319 (2005)). In addition, it has been reported that the strong effect of the anticancer drug imatinib (Gleevec) on solid gastrointestinal stromal tumor is derived from the inhibition of IDO (Balachandran, V. P. et al. Nature Med. 17, 1094-1100 (2011)).

Therefore, IDO inhibitors can effectively inhibit cancer metastasis and cancer proliferation. And, IDO inhibitors can be also usefully applied for the treatment and prevention of viral infections and autoimmune diseases such as rheumatoid arthritis. In addition, IDO inhibitors can be used to activate T cells, during the pregnancy, malignant tumors, or virus-induced T cell suppression. Although the mechanism of action is not well defined, it is expected that IDO inhibitors can be also applied for the treatment of patients with neuropsychiatric diseases or symptoms such as depression. For example, WO 2016/073770, WO 2018/039512, etc. have disclosed a compound having inhibitory activity against indoleamine 2,3-dioxygenase and a pharmaceutical composition comprising the same.

### DISCLOSURE

### Technical Problem

The present inventors have found that a derivative having a cyclohexyl-(alkyl or cycloalkyl-substituted)ethylene-amino-heteroaryl moiety or pharmaceutically acceptable salt thereof not only has excellent inhibitory activity against indoleamine 2,3-dioxygenase but also exhibits remarkably high in vivo exposure upon oral administration. Therefore, the derivative or pharmaceutically acceptable salt thereof can be usefully applied for preventing or treating various diseases associated with IDO, e.g., proliferative disorders such as cancer, viral infections and/or autoimmune diseases, etc.

Therefore, the present invention provides said derivative having a cyclohexyl-(alkyl or cycloalkyl-substituted)ethylene-amino-heteroaryl moiety or pharmaceutically to acceptable salt thereof, a process for the preparation thereof, a pharmaceutical composition comprising the same, and a use thereof.

### Technical Solution

According to an aspect of the present invention, there is provided a derivative having a cyclohexyl-(alkyl or cycloalkyl-substituted)ethylene-amino-heteroaryl moiety or pharmaceutically acceptable salt thereof.

According to another aspect of the present invention, there is provided a process for preparing said derivative having a cyclohexyl-(alkyl or cycloalkyl-substituted)ethylene-amino-heteroaryl moiety or pharmaceutically acceptable salt thereof.

According to still another aspect of the present invention, there is provided a pharmaceutical composition comprising said derivative having a cyclohexyl-(alkyl or cycloalkyl-substituted)ethylene-amino-heteroaryl moiety or pharmaceutically acceptable salt thereof as an active ingredient.

According to still another aspect of the present invention, there is provided a therapeutic method comprising administering said derivative having a cyclohexyl-(alkyl or cycloalkyl-substituted)ethylene-amino-heteroaryl moiety or pharmaceutically acceptable salt thereof.

According to still another aspect of the present invention, there is provided a use of said derivative having a cyclohexyl-(alkyl or cycloalkyl-substituted)ethylene-amino-heteroaryl moiety or pharmaceutically acceptable salt thereof for the manufacture of a medicament for inhibiting indoleamine 2,3-dioxygenase.

### Advantageous Effects

It has been found by the present invention that the derivative having a cyclohexyl-(alkyl or cycloalkyl-substituted)ethylene-amino-heteroaryl moiety or pharmaceutically acceptable salt thereof not only has excellent inhibitory activity against indoleamine 2,3-dioxygenase but also exhibits remarkably high in vivo exposure upon oral administration. Therefore, the compound or pharmaceutically acceptable salt thereof according to the present invention can be usefully applied for preventing or treating various diseases associated with indoleamine 2,3-dioxygenase, e.g., proliferative disorders such as cancer, viral infections and/or autoimmune diseases, etc.

### DESCRIPTION OF DRAWINGS

FIG. 1 shows the blood concentration profiles obtained from the oral administrations of the compounds of the present invention and the control (BMS-986205) to rats.

### BEST MODE FOR CARRYING OUT THE INVENTION

The present invention provides a compound or salt thereof having excellent inhibitory activity against indoleamine 2,3-dioxygenase, i.e., a compound of Formula 1 or pharmaceutically acceptable salt thereof:

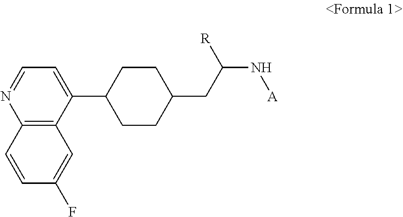

wherein,

R is a C₁˜C₆ alkyl group or a C₃˜C₁₀ cycloalkyl group,

A is a heteroaryl group selected from the group consisting of quinazolinyl, 2H-chromen-2-on-yl, benzothiazolyl, benzoxazolyl, thiazolopyridinyl, oxazolopyridinyl, isoquinolinyl, and phthalazinyl, wherein the heteroaryl group is optionally substituted with one or two substituents selected from the group consisting of halogen, C₁˜C₆ alkyl, trifluoromethyl, C₁˜C₆ alkoxy, halogeno-C₁˜C₆ alkoxy, trifluoromethoxy, and cyano.

In an embodiment of the present invention, R may be methyl, ethyl, or cyclopropyl.

In another embodiment of the present invention, A may be a substituted or unsubstituted quinazolinyl group, preferably a quinazolinyl group substituted with one or two substituents selected from the group consisting of halogen, C₁˜C₆ alkyl, trifluoromethyl, C₁˜C₆ alkoxy, trifluoromethoxy, and cyano. For example, A may have the following structure of Formula 1a.

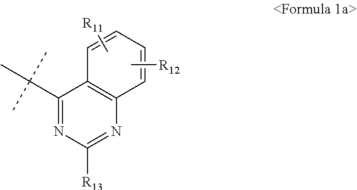

wherein R₁₁ and R₁₂ are, independently each other, selected from the group consisting of hydrogen, halogen, C₁˜C₆ alkyl, trifluoromethyl, C₁˜C₆ alkoxy, halogeno-C₁˜C₆ alkoxy, trifluoromethoxy, and cyano; and R₁₃ may be hydrogen or halogen. In an embodiment, R₁₃ may be hydrogen, Cl, or F.

In still another embodiment of the present invention, A may be a substituted or unsubstituted 2H-chromen-2-on-yl group, preferably a 2H-chromen-2-on-yl substituted with one or two halogens. For example, A may have the following structure of Formula 1b.

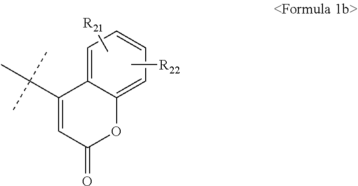

wherein R₂₁ and R₂₂ are, independently each other, selected from the group consisting of hydrogen, halogen, C₁˜C₆ alkyl, trifluoromethyl, C₁˜C₆ alkoxy, halogeno-C₁˜C₆ alkoxy, trifluoromethoxy, and cyano.

In still another embodiment of the present invention, A may be a substituted or unsubstituted benzothiazolyl group, preferably a benzothiazolyl substituted with one or two substituents selected from the group consisting of halogen and C₁˜C₆ alkoxy. For example, A may have the following structure of Formula 1c.

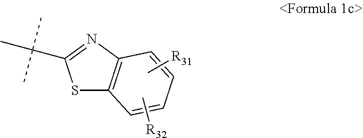

wherein R₃₁ and R₃₂ are, independently each other, selected from the group consisting of hydrogen, halogen, C₁˜C₆ alkyl, trifluoromethyl, C₁˜C₆ alkoxy, halogeno-C₁˜C₆ alkoxy, trifluoromethoxy, and cyano.

In still another embodiment of the present invention, A may be a substituted or unsubstituted thiazolopyridinyl group, preferably a thiazolopyridinyl optionally substituted with halogen or C₁˜C₆ alkoxy. For example, A may have the following structure of Formula 1d-1 or 1d-2.

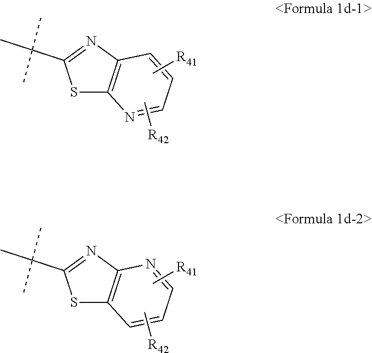

wherein R₄₁ and R₄₂ are, independently each other, selected from the group consisting of hydrogen, halogen, C₁˜C₆ alkyl, trifluoromethyl, C₁˜C₆ alkoxy, halogeno-C₁˜C₆ alkoxy, trifluoromethoxy, and cyano.

In still another embodiment of the present invention, A may be a substituted or unsubstituted benzoxazolyl group, preferably a benzoxazolyl optionally substituted with one or two substituents selected from the group consisting of halogen, C₁˜C₆ alkoxy, and halogeno-C₁˜C₆ alkoxy. For example, A may have the following structure of Formula 1e.

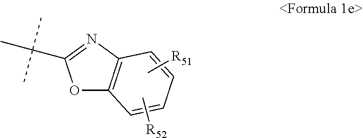

wherein R₅₁ and R₅₂ are, independently each other, selected from the group consisting of hydrogen, halogen, C₁˜C₆ alkyl, trifluoromethyl, C₁˜C₆ alkoxy, halogeno-C₁˜C₆ alkoxy, trifluoromethoxy, and cyano.

In still another embodiment of the present invention, A may be a substituted or unsubstituted oxazolopyridinyl group, preferably an oxazolopyridinyl group optionally substituted with halogen. For example, A may have the following structure of Formula 1f-1 or 1f-2.

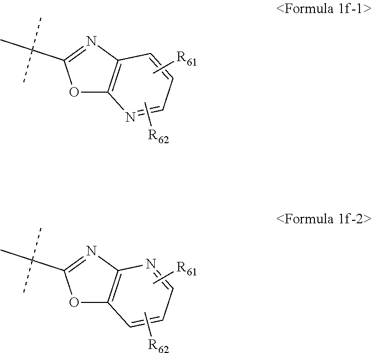

wherein R₆₁ and R₆₂ are, independently each other, selected from the group consisting of hydrogen, halogen, C₁˜C₆ alkyl, trifluoromethyl, C₁˜C₆ alkoxy, halogeno-C₁˜C₆ alkoxy, trifluoromethoxy, and cyano.

In still another embodiment of the present invention, A may be a substituted or unsubstituted isoquinolinyl group, preferably an isoquinolinyl group substituted with halogen. For example, A may have the following structure of Formula 1g.

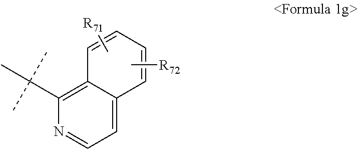

wherein R₇₁ and R₇₂ are, independently each other, selected from the group consisting of hydrogen, halogen, C₁˜C₆ alkyl, trifluoromethyl, C₁˜C₆ alkoxy, halogeno-C₁˜C₆ alkoxy, trifluoromethoxy, and cyano.

In still another embodiment of the present invention, A may be a substituted or unsubstituted phthalazinyl group, preferably a phthalazinyl group substituted with halogen. For example, A may have the following structure of Formula 1h.

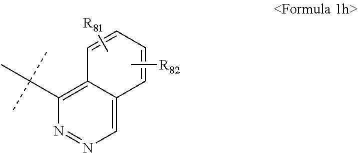

wherein R₈₁ and R₈₂ are, independently each other, selected from the group consisting of hydrogen, halogen, C₁˜C₆ alkyl, trifluoromethyl, C₁˜C₆ alkoxy, halogeno-C₁˜C₆ alkoxy, trifluoromethoxy, and cyano.

In the present specification, when the heteroaryl is substituted with two halogens, the halogens may be the same or different. When the heteroaryl is substituted with two C₁˜C₆ alkyl groups, the C₁˜C₆ alkyl groups may be the same or different. When the heteroaryl is substituted with two C₁˜C₆ alkoxy groups, the C₁˜C₆ alkoxy groups may be the same or different. When the heteroaryl is substituted with two halogeno-C₁˜C₆ alkoxy groups, the halogeno-C₁˜C₆ alkoxy groups may be the same or different.

In the compound of Formula 1 or pharmaceutically acceptable salt, preferable compounds include a compound or pharmaceutically acceptable salt thereof selected from the group consisting of:
4-(((R)-1-((cis)-4-(6-fluoroquinolin-4-yl)cyclohexyl)propan-2-yl)amino)quinazolin-7-carbonitrile,
7-bromo-N-((R)-1-((cis)-4-(6-fluoroquinolin-4-yl)cyclohexyl)propan-2-yl)quinazolin-4-amine,
7-chloro-N-((R)-1-((cis)-4-(6-fluoroquinolin-4-yl)cyclohexyl)propan-2-yl)quinazolin-4-amine,
7-chloro-N-((R)-1-((trans)-4-(6-fluoroquinolin-4-yl)cyclohexyl)propan-2-yl)quinazolin-4-amine;
7-bromo-4-(((R)-1-((trans)-4-(6-fluoroquinolin-4-yl)cyclohexyl)propan-2-yl)amino)-2H-chromen-2-one,
7-chloro-4-(((R)-1-((trans)-4-(6-fluoroquinolin-4-yl)cyclohexyl)propan-2-yl)amino)-2H-chromen-2-one,
6-bromo-4-(((R)-1-((trans)-4-(6-fluoroquinolin-4-yl)cyclohexyl)propan-2-yl)amino)-2H-chromen-2-one,
7-chloro-4-(((R)-1-((cis)-4-(6-fluoroquinolin-4-yl)cyclohexyl)propan-2-yl)amino)-2H-chromen-2-one,
7-bromo-4-(((R)-1-((cis)-4-(6-fluoroquinolin-4-yl)cyclohexyl)propan-2-yl)amino)-2H-chromen-2-one;
7-chloro-3-fluoro-4-(((R)-1-((trans)-4-(6-fluoroquinolin-4-yl)cyclohexyl)propan-2-yl)amino)-2H-chromen-2-one,
7-bromo-3-fluoro-4-(((R)-1-((cis)-4-(6-fluoroquinolin-4-yl)cyclohexyl)propan-2-yl)amino)-2H-chromen-2-one,
7-bromo-3-fluoro-4-(((R)-1-((trans)-4-(6-fluoroquinolin-4-yl)cyclohexyl)propan-2-yl)amino)-2H-chromen-2-one,
6-chloro-N-((R)-1-((cis)-4-(6-fluoroquinolin-4-yl)cyclohexyl)propan-2-yl)benzo[d]thiazol-2-amine,
N-((R)-1-((cis)-4-(6-fluoroquinolin-4-yl)cyclohexyl)propan-2-yl)-6-methoxybenzo[d]thiazol-2-amine;
N-((R)-1-((cis)-4-(6-fluoroquinolin-4-yl)cyclohexyl)butan-2-yl)-6-methoxybenzo[d]thiazol-2-amine,
N-((S)-1-cyclopropyl-2-((cis)-4-(6-fluoroquinolin-4-yl)cyclohexyl)ethyl)-6-methoxybenzo[d]thiazol-2-amine,
4,6-difluoro-N((R)-1-((cis)-4-(6-fluoroquinolin-4-yl)cyclohexyl)propan-2-yl)benzo[d]thiazol-2-amine,
7-chloro-N-((R)-1-((cis)-4-(6-fluoroquinolin-4-yl)cyclohexyl)propan-2-yl)benzo[d]thiazol-2-amine,
6-ethoxy-N-((R)-1-((cis)-4-(6-fluoroquinolin-4-yl)cyclohexyl)propan-2-yl)benzo[d]thiazol-2-amine;
6-ethoxy-N-((R)-1-((cis)-4-(6-fluoroquinolin-4-yl)cyclohexyl)butan-2-yl)benzo[d]thiazol-2-amine,
N-((R)-1-((cis)-4-(6-fluoroquinolin-4-yl)cyclohexyl)propan-2-yl)thiazolo[4,5-b]pyridin-2-amine,
N-((R)-1-((cis)-4-(6-fluoroquinolin-4-yl)cyclohexyl)propan-2-yl)-5-methoxythiazolo[5,4-b]pyridin-2-amine,
6-chloro-N-((R)-1-((cis)-4-(6-fluoroquinolin-4-yl)cyclohexyl)propan-2-yl)thiazolo[4,5-b]pyridin-2-amine,
N-((R)-1-((cis)-4-(6-fluoroquinolin-4-yl)cyclohexyl)propan-2-yl)benzo[d]oxazol-2-amine;
6-chloro-N-((R)-1-((cis)-4-(6-fluoroquinolin-4-yl)cyclohexyl)propan-2-yl)benzo[d]oxazol-2-amine,
N-((R)-1-((cis)-4-(6-fluoroquinolin-4-yl)cyclohexyl)propan-2-yl)-6-methoxybenzo[d]oxazol-2-amine,
6-chloro-N-((R)-1-((cis)-4-(6-fluoroquinolin-4-yl)cyclohexyl)butan-2-yl)benzo[d]oxazol-2-amine,
4-fluoro-N-((R)-1-((cis)-4-(6-fluoroquinolin-4-yl)cyclohexyl)propan-2-yl)benzo[d]oxazol-2-amine;
6-fluoro-N-((R)-1-((cis)-4-(6-fluoroquinolin-4-yl)cyclohexyl)propan-2-yl)benzo[d]oxazol-2-amine,
N-((R)-1-((cis)-4-(6-fluoroquinolin-4-yl)cyclohexyl)propan-2-yl)oxazolo[5,4-b]pyridin-2-amine;
6-chloro-N-((R)-1-((cis)-4-(6-fluoroquinolin-4-yl)cyclohexyl)propan-2-yl)oxazolo[4,5-b]pyridin-2-amine,
N-((R)-1-((cis)-4-(6-fluoroquinolin-4-yl)cyclohexyl)propan-2-yl)oxazolo[4,5-b]pyridin-2-amine,
6-fluoro-N-((R)-1-((cis)-4-(6-fluoroquinolin-4-yl)cyclohexyl)propan-2-yl)isoquinolin-1-amine,
6-chloro-N-((R)-1-((cis)-4-(6-fluoroquinolin-4-yl)cyclohexyl)propan-2-yl)isoquinolin-1-amine,
4-chloro-N-((R)-1-((cis)-4-(6-fluoroquinolin-4-yl)cyclohexyl)propan-2-yl)benzo[d]thiazol-2-amine,
5-fluoro-N-((R)-1-((cis)-4-(6-fluoroquinolin-4-yl)cyclohexyl)propan-2-yl)benzo[d]oxazol-2-amine,
7-fluoro-N-((R)-1-((cis)-4-(6-fluoroquinolin-4-yl)cyclohexyl)propan-2-yl)benzo[d]oxazol-2-amine,
2-chloro-N-((R)-1-((cis)-4-(6-fluoroquinolin-4-yl)cyclohexyl)propan-2-yl)quinazolin-4-amine,
6-chloro-N-((R)-1-((cis)-4-(6-fluoroquinolin-4-yl)cyclohexyl)propan-2-yl)phthalazin-1-amine,
2,7-dichloro-N-((R)-1-((cis)-4-(6-fluoroquinolin-4-yl)cyclohexyl)propan-2-yl)quinazolin-4-amine;
2,7-dichloro-N-((R)-1-((trans)-4-(6-fluoroquinolin-4-yl)cyclohexyl)propan-2-yl)quinazolin-4-amine,
7-fluoro-N-((R)-1-((cis)-4-(6-fluoroquinolin-4-yl)cyclohexyl)propan-2-yl)-6-methoxybenzo[d]oxazol-2-amine,
6-chloro-5-fluoro-N((R)-1-((cis)-4-(6-fluoroquinolin-4-yl)cyclohexyl)propan-2-yl)benzo[d]oxazol-2-amine,
6-ethoxy-N-((R)-1-((cis)-4-(6-fluoroquinolin-4-yl)cyclohexyl)propan-2-yl)benzo[d]oxazol-2-amine;
6-(2-fluoroethoxy)-N-((R)-1-((cis)-4-(6-fluoroquinolin-4-yl)cyclohexyl)propan-2-yl)benzo[d]oxazol-2-amine,
6,7-dichloro-N-((R)-1-((cis)-4-(6-fluoroquinolin-4-yl)cyclohexyl)propan-2-yl)benzo[d]oxazol-2-amine;
6,7-difluoro-N-((R)-1-((cis)-4-(6-fluoroquinolin-4-yl)cyclohexyl)propan-2-yl)benzo[d]oxazol-2-amine,
2-chloro-6-fluoro-N-((R)-1-((trans)-4-(6-fluoroquinolin-4-yl)cyclohexyl)propan-2-yl)quinazolin-4-amine,
2,6-dichloro-N-((R)-1-((trans)-4-(6-fluoroquinolin-4-yl)cyclohexyl)propan-2-yl)quinazolin-4-amine,
2-chloro-8-fluoro-N-((R)-1-((trans)-4-(6-fluoroquinolin-4-yl)cyclohexyl)propan-2-yl)quinazolin-4-amine,
2,8-dichloro-N-((R)-1-((trans)-4-(6-fluoroquinolin-4-yl)cyclohexyl)propan-2-yl)quinazolin-4-amine;
2-chloro-N-((R)-1-((trans)-4-(6-fluoroquinolin-4-yl)cyclohexyl)propan-2-yl)-8-methoxyquinazolin-4-amine,
2-chloro-N-((R)-1-((trans)-4-(6-fluoroquinolin-4-yl)cyclohexyl)propan-2-yl)-8-(trifluoromethoxy)quinazolin-4-amine,
2-chloro-7-fluoro-N-((R)-1-((trans)-4-(6-fluoroquinolin-4-yl)cyclohexyl)propan-2-yl)quinazolin-4-amine,
2-chloro-4-(((R)-1-((trans)-4-(6-fluoroquinolin-4-yl)cyclohexyl)propan-2-yl)amino)quinazolin-7-carbonitrile,
7-bromo-2-chloro-N-((R)-1-((trans)-4-(6-fluoroquinolin-4-yl)cyclohexyl)propan-2-yl)quinazolin-4-amine,
2-chloro-N-((R)-1-((trans)-4-(6-fluoroquinolin-4-yl)cyclohexyl)propan-2-yl)-7-methylquinazolin-4-amine,
2-chloro-N-((R)-1-((trans)-4-(6-fluoroquinolin-4-yl)cyclohexyl)propan-2-yl)-7-(trifluoromethyl)quinazolin-4-amine,
2-chloro-N-((R)-1-((trans)-4-(6-fluoroquinolin-4-yl)cyclohexyl)propan-2-yl)-7-methoxyquinazolin-4-amine,
2-chloro-6-fluoro-N-((R)-1-((cis)-4-(6-fluoroquinolin-4-yl)cyclohexyl)propan-2-yl)quinazolin-4-amine,
2,6-dichloro-N-((R)-1-((cis)-4-(6-fluoroquinolin-4-yl)cyclohexyl)propan-2-yl)quinazolin-4-amine,
2-chloro-7-fluoro-N-((R)-1-((cis)-4-(6-fluoroquinolin-4-yl)cyclohexyl)propan-2-yl)quinazolin-4-amine;
2-chloro-4-(((R)-1-((cis)-4-(6-fluoroquinolin-4-yl)cyclohexyl)propan-2-yl)amino)quinazolin-7-carbonitrile,
7-bromo-2-chloro-N-((R)-1-((cis)-4-(6-fluoroquinolin-4-yl)cyclohexyl)propan-2-yl)quinazolin-4-amine,
2-chloro-N-((R)-1-((cis)-4-(6-fluoroquinolin-4-yl)cyclohexyl)propan-2-yl)-7-methylquinazolin-4-amine,
2-chloro-N-((R)-1-((cis)-4-(6-fluoroquinolin-4-yl)cyclohexyl)propan-2-yl)-7-(trifluoromethyl)quinazolin-4-amine,
2-chloro-N-((R)-1-((cis)-4-(6-fluoroquinolin-4-yl)cyclohexyl)propan-2-yl)-7-methoxyquinazolin-4-amine,
2,8-dichloro-N-((R)-1-((cis)-4-(6-fluoroquinolin-4-yl)cyclohexyl)propan-2-yl)quinazolin-4-amine,
2-chloro-8-fluoro-N-((R)-1-((cis)-4-(6-fluoroquinolin-4-yl)cyclohexyl)propan-2-yl)quinazolin-4-amine,
2-chloro-N-((R)-1-((cis)-4-(6-fluoroquinolin-4-yl)cyclohexyl)propan-2-yl)-8-methoxyquinazolin-4-amine, and
2-chloro-N-((R)-1-((cis)-4-(6-fluoroquinolin-4-yl)cyclohexyl)propan-2-yl)-8-(trifluoromethoxy)quinazolin-4-amine.

In the compound of Formula 1 or pharmaceutically acceptable salt, more preferable compounds include a compound or pharmaceutically acceptable salt thereof selected from the group consisting of:
7-bromo-N-((R)-1-((cis)-4-(6-fluoroquinolin-4-yl)cyclohexyl)propan-2-yl)quinazolin-4-amine,
7-bromo-4-(((R)-1-((trans)-4-(6-fluoroquinolin-4-yl)cyclohexyl)propan-2-yl)amino)-2H-chromen-2-one;
6-chloro-N-((R)-1-((cis)-4-(6-fluoroquinolin-4-yl)cyclohexyl)propan-2-yl)benzo[d]oxazol-2-amine,
6-chloro-N-((R)-1-((cis)-4-(6-fluoroquinolin-4-yl)cyclohexyl)butan-2-yl)benzo[d]oxazol-2-amine,
4-fluoro-N-((R)-1-((cis)-4-(6-fluoroquinolin-4-yl)cyclohexyl)propan-2-yl)benzo[d]oxazol-2-amine,
6-fluoro-N-((R)-1-((cis)-4-(6-fluoroquinolin-4-yl)cyclohexyl)propan-2-yl)benzo[d]oxazol-2-amine, and
6,7-difluoro-N-((R)-1-((cis)-4-(6-fluoroquinolin-4-yl)cyclohexyl)propan-2-yl)benzo[d]oxazol-2-amine.

In the compound of Formula 1 or pharmaceutically acceptable salt, especially preferable compounds include 6-chloro-N-((R)-1-((cis)-4-(6-fluoroquinolin-4-yl)cyclohexyl)propan-2-yl)benzo[d]oxazol-2-amine or pharmaceutically acceptable salt thereof.

The compound of Formula 1 or pharmaceutically acceptable salt thereof may have a substituent including an asymmetric atom and may have geometric isomers via cyclohexyl. That is, the compound of Formula 1 or pharmaceutically acceptable salt thereof may be in the form of cis- or trans-geometrical isomer; in the form of (R)- or (S)-optical isomer; or in the form of racemic mixture (RS). Therefore, the compound of Formula 1 or pharmaceutically acceptable salt thereof includes a cis- or trans-geometrical isomer, a (R)- or (S)-optical isomer, and a racemic mixture (RS), unless otherwise indicated.

The compound of Formula 1 of the present invention may be in a pharmaceutically acceptable salt form. The salt may be a conventional acid addition salt form, which includes e.g., salts derived from an inorganic acid such as hydrochloric acid, hydrobromic acid, sulfuric acid, sulfamic acid, phosphoric acid or nitric acid; and salts derived from an organic acid such as acetic acid, propionic acid, succinic acid, glycolic acid, stearic acid, citric acid, maleic acid, malonic acid, methanesulfonic acid, tartaric acid, malic acid, phenylacetic acid, glutamic acid, benzoic acid, salicylic acid, 2-acetoxybenzoic acid, fumaric acid, p-toluenesulfonic acid, oxalic acid or trifluoroacetic acid. In addition, the salt includes conventional metal salt forms, e.g., salts derived from metals such as lithium, sodium, potassium, magnesium, or calcium. The acid addition salt or metal salt may be prepared according to conventional methods.

The present invention includes, within its scope, a process for preparing the compound of Formula 1 or pharmaceutically acceptable salt thereof.

For example, the compound of Formula 1 or pharmaceutically acceptable salt thereof according to the present invention may be prepared by a process which comprises reacting a compound of Formula 2 or salt thereof with a compound of Formula 3 to obtain a compound of Formula 1; and optionally converting the compound of Formula 1 to a pharmaceutically acceptable salt thereof:

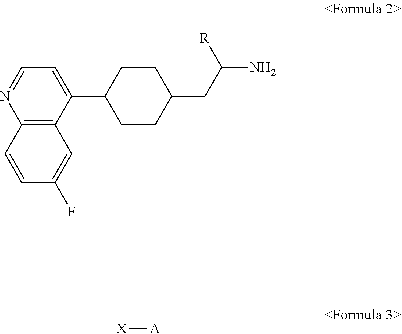

wherein, R and A are the same as defined in the above; and X is halogen.

The compound of Formula 3 is commercially available. The coupling reaction between the compound of Formula 2 or salt thereof (e.g., hydrochloride) and the compound of Formula 3 may be carried out in the presence of a base and a solvent. The base may be cesium carbonate, potassium carbonate, sodium carbonate, triethylamine, and the like, and the solvent may be an organic solvent such as N,N-dimethylformamide, 1,4-dioxane, tetrahydrofuran, ethanol, or isopropyl alcohol. In addition, the reaction may be carried out at room temperature to 100° C.

The compound of Formula 2 or salt thereof, which is a novel compound, may be usefully used as an intermediate in the preparation of the derivative having a cyclohexyl-(alkyl or cycloalkyl-substituted)ethylene-amino-heteroaryl moiety or pharmaceutically acceptable salt thereof according to the present invention (i.e., the compound of Formula 1 or pharmaceutically acceptable salt thereof). Therefore, the present invention includes, within its scope, the compound of Formula 2 or salt thereof. The salt of the compound of Formula 2 includes an acid addition salt such as hydrochloride.

For example, the compound of Formula 2a (R=methyl) may be prepared according to the following Reaction Scheme 1.

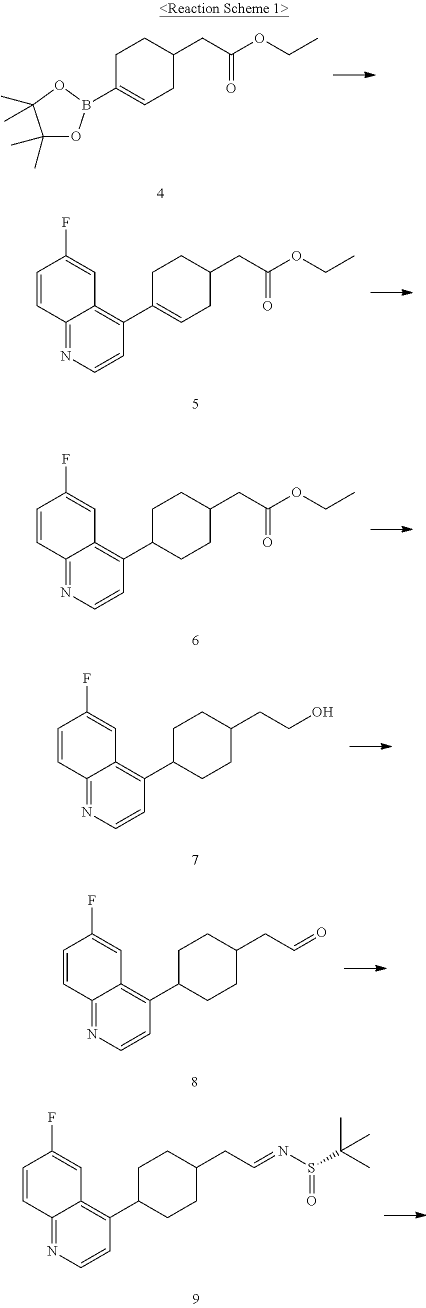 [SMILES: CCOC(=O)CC1CC=C(B2OC(C)(C)C(C)(C)O2)CC1 ; CCOC(=O)CC1CC=C(c2ccnc3ccc(F)cc23)CC1 ; CCOC(=O)CC1CCC(c2ccnc3ccc(F)cc23)CC1 ; OCCC1CCC(c2ccnc3ccc(F)cc23)CC1 ; O=CCC1CCC(c2ccnc3ccc(F)cc23)CC1 ; CC(C)(C)[S@](=O)/N=C/CC1CCC(c2ccnc3ccc(F)cc23)CC1]

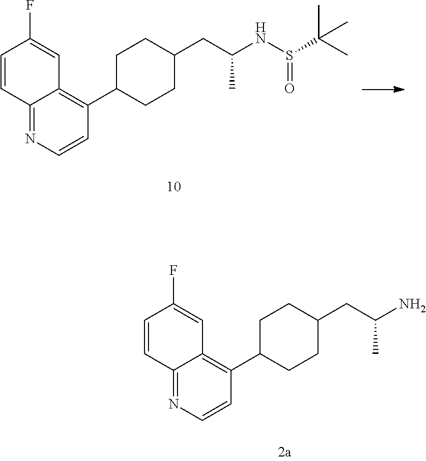 [SMILES: C[C@H](CC1CCC(c2ccnc3ccc(F)cc23)CC1)N[S@@](=O)C(C)(C)C ; C[C@@H](N)CC1CCC(c2ccnc3ccc(F)cc23)CC1]

The compound of Formula 5 may be prepared through the Suzuki reaction between the compound of Formula 4 (which is commercially available) and 4-chloro-6-fluoroquinoline. The reaction may be carried out using a palladium catalyst, such as palladium(II) acetate (Pd(OAc)₂), tris(dibenzylideneacetone)dipalladium (Pd₂(dba)₃), tetrakis(triphenylphosphine)palladium(0) (Pd(PPh₃)₄), [1,1′-bis(diphenylphosphino)ferrocene]dichloropalladium(II) (Pd(dppf)Cl₂), etc. In addition, said reaction may be carried out in the presence of a ligand and a base, in addition to the palladium catalyst. The ligand includes (S)-2,2′-bis(diphenylphosphino)-1,1′-binaphthyl (BINAP), 1,1′-bis(diphenylphosphino)ferrocene (dppf), tri(o-tolyl)phosphine (P(o-Tol)₃), etc. The base includes an inorganic base such as cesium carbonate (Cs₂CO₃), sodium carbonate (Na₂CO₃), potassium carbonate (K₂CO₃), potassium fluoride (KF), cesium fluoride (CsF), sodium hydroxide (NaOH), potassium phosphate (K₃PO₄), sodium tert-butoxide (tert-BuONa), potassium tert-butoxide (tert-BuOK) etc. Said reaction may be carried out in a non-polar organic solvent such as benzene or toluene or in a polar organic solvent such as 1,4-dioxane, tetrahydrofuran, acetonitrile, 1,2-dimethoxyethane, or N,N-dimethylformamide, at 50° C. to 150° C., preferably 80° C. to 110° C. Other reaction conditions including a reaction time may be determined according to known methods for the Suzuki reaction (Barbara Czako and Laszlo Kurti, STRATEGIC APPLICATIONS of NAMED REACTIONS in ORGANIC SYNTHESIS, 2005).

The reduction of the compound of Formula 5 may be carried out using palladium/carbon in an organic solvent such as ethyl acetate or methanol. The reduction may be carried out typically at room temperature using hydrogen.

The reduction of the compound of Formula 6 may be carried out using lithium aluminium hydride in an organic solvent such as tetrahydrofuran or dichloromethane. The reduction may be carried out typically at −78° C. to room temperature.

The oxidation of the compound of Formula 7 may be carried out using an oxidizing agent in an organic solvent such as ethyl acetate or dichloromethane. The oxidation may be carried out typically at 0° C. to room temperature.

The compound of Formula 9 may be prepared by condensing the compound of Formula 8 with (S)-(−)-2-methyl-2-propanesulfinamide. The condensation may be carried out in an organic solvent such as ethyl acetate, dichloromethane, or tetrahydrofuran in the presence of a Lewis acid catalyst such as titanium(IV) isopropoxide or titanium(IV) epoxide. The reaction may be carried out at −78° C. to room temperature.

The compound of Formula 10 may be prepared by reacting the compound of Formula 9 with an alkyl Grignard reagent. In addition, deprotection of the compound of Formula 10 may give the compound of Formula 2a or salt thereof (eg, hydrochloride). The deprotection may be carried out according to a known method (Theodora W. Greene and Peter G. M. Wuts, Protective groups in organic synthesis, 3rd Ed., 1999). For example, the deprotection may be carried out using a trifluoroacetic acid or hydrochloric acid solution, in an organic solvent such as dichloromethane, 1,4-dioxane, or ethyl acetate, at room temperature.

The derivative having a cyclohexyl-(alkyl or cycloalkyl-substituted)ethylene-amino-heteroaryl moiety or pharmaceutically acceptable salt thereof according to the present invention (i.e., the compound of Formula 1 or pharmaceutically acceptable salt thereof) has excellent inhibitory activity against indoleamine 2,3-dioxygenase (IDO). And, the compound of Formula 1 or pharmaceutically acceptable salt thereof exhibits remarkably high in vivo exposure upon oral administration. The present inventors carried out the studies on biomarker changes and pharmacokinetics in the subcutaneous MC38 colorectal model of C57BL/6 mice. When orally administered for 1 day, the groups administered with the compounds of the present invention showed significantly reduced kynurenine level in the plasma and tumor tissue, in comparison with the vehicle group and the positive control groups (BMS-986205, Epacadostat). When orally administered for 3 days, the groups administered with the compounds of the present invention showed remarkably reduced kynurenine level in the plasma and tumor tissue. Especially, in the group administered with a certain compound of the present invention for 3 days, the level of kynurenine in the plasma and tumor tissue was almost completely reduced. Therefore, the compound of Formula 1 or pharmaceutically acceptable salt thereof can be usefully applied for preventing or treating various diseases associated with IDO.

Therefore, the present invention includes, within its scope, a pharmaceutical composition for inhibiting indoleamine 2,3-dioxygenase comprising a therapeutically effective amount of the compound of Formula 1 or pharmaceutically acceptable salt thereof as an active ingredient. In an embodiment, the present invention provides a pharmaceutical composition for preventing or treating the diseases associated with IDO, such as viral infection; autoimmune disease (e.g., rheumatoid arthritis); cancer (e.g., melanoma, pancreatic cancer, prostate cancer, brain cancer); or neuropsychiatric disease (e.g., depression), comprising a therapeutically effective amount of the compound of Formula 1 or pharmaceutically acceptable salt thereof as an active ingredient.

The pharmaceutical composition of the present invention may comprise a pharmaceutically acceptable carrier, such as diluents, disintegrants, sweeteners, lubricants, or flavoring agents, which is conventionally used in the art. The pharmaceutical composition may be formulated to an oral dosage form such as tablets, capsules, powders, granules, suspensions, emulsions, or syrups; or a parenteral dosage form such as solutions for external use, suspensions for external use, emulsions for external use, gels (e.g., ointment), inhalations, nebulizations, injections, according to conventional methods. The dosage form may be various forms, e.g., dosage forms for single administration or for multiple administrations.

The pharmaceutical composition of the present invention may comprise, for example, a diluent (e.g., lactose, corn starch, etc); a lubricant (e.g., magnesium stearate); an emulsifying agent; a suspending agent; a stabilizer; and/or an isotonic agent. If necessary, the composition further comprises sweeteners and/or flavoring agents.

The composition of the present invention may be administered orally or parenterally, including inhalant, intravenous, intraperitoneal, subcutaneous, rectal and topical routes of administration. Therefore, the composition of the present invention may be formulated into various forms such as tablets, capsules, aqueous solutions or suspensions. In the case of tablets for oral administration, carriers such as lactose, corn starch, and lubricating agents, e.g. magnesium stearate, are conventionally used. In the case of capsules for oral administration, lactose and/or dried corn starch can be used as a diluent. When an aqueous suspension is required for oral administration, the active ingredient may be combined with emulsifying and/or suspending agents. If desired, certain sweetening and/or flavoring agents may be used. For intramuscular, intraperitoneal, subcutaneous and intravenous administration, sterile solutions of the active ingredient are usually prepared, and the pH of the solutions should be suitably adjusted and buffered. For intravenous administration, the total concentration of solutes should be controlled in order to render the preparation isotonic. The composition of the present invention may be in the form of an aqueous solution containing pharmaceutically acceptable carriers, e.g., saline having a pH level of 7.4. The solutions may be introduced into a patient's intramuscular blood-stream by local bolus injection.

The compound of Formula 1 or pharmaceutically acceptable salt thereof may be singly or multiply administered in a therapeutically effective amount ranging from about 5 mg/kg to about 50 mg/kg per day, preferably from about 10 mg/kg to about 20 mg/kg per day, to a subject patient. Of course, the dosage may be changed according to the patient's age, weight, susceptibility, symptom, or activity of the compound.

The present invention includes, within its scope, a method for inhibiting indoleamine 2,3-dioxygenase in a mammal, comprising administering a therapeutically effective amount of the compound of Formula 1 or pharmaceutically acceptable salt thereof to the mammal in need thereof. In an embodiment, the present invention provides a method for treating the diseases associated with indoleamine 2,3-dioxygenase, such as viral infection; autoimmune disease (e.g., rheumatoid arthritis); cancer (e.g., melanoma, pancreatic cancer, prostate cancer, brain cancer); or neuropsychiatric disease (e.g., depression), comprising administering a therapeutically effective amount of the compound of Formula 1 or pharmaceutically acceptable salt thereof to the mammal in need thereof.

The present invention also provides a use of the compound of Formula 1 or pharmaceutically acceptable salt thereof for the manufacture of a medicament for inhibiting indoleamine 2,3-dioxygenase in a mammal. In an embodiment, the present invention provides a use of the compound of Formula 1 or pharmaceutically acceptable salt thereof for the manufacture of a medicament for preventing or treating the diseases associated with indoleamine 2,3-dioxygenase, such as viral infection; autoimmune disease (e.g., rheumatoid arthritis); cancer (e.g., melanoma, pancreatic cancer, prostate cancer, brain cancer); or neuropsychiatric disease (e.g., depression).

The following examples and experimental examples are provided for illustration purposes only, and are not intended to limit the scope of the invention.

The analyses of the compounds prepared in the following Examples were carried out as follows: Nuclear magnetic resonance (NMR) spectrum analysis was carried out using Bruker 400 MHz spectrometer and chemical shifts thereof were analyzed in ppm. Column chromatography was carried out on silica gel (Merck, 70-230 mesh) (W. C. Still, J. Org. Chem., 43, 2923, 1978). Each starting material is a known compound which was synthesized according to literatures or purchased commercially, e.g., from Sigma-Aldrich.

### Preparation 1. (R)-1-((cis)-4-(6-fluoroquinolin-4-yl)cyclohexyl)propan-2-amine hydrochloride

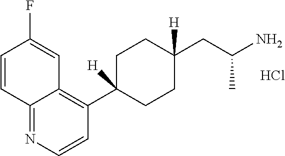 [SMILES: C[C@@H](N)C[C@H]1CC[C@@H](c2ccnc3ccc(F)cc32)CC1 ; Cl]

### Step 1: ethyl 2-(4-(6-fluoroquinolin-4-yl)cyclohex-3-en-1-yl)acetate

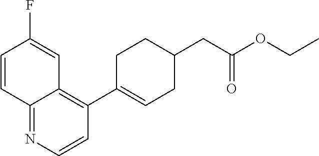 [SMILES: CCOC(=O)CC1CC=C(c2ccnc3ccc(F)cc23)CC1]

Ethyl 2-(4-(4,4,5,5-tetramethyl-1,3,2-dioxaborolan-2-yl)cyclohex-3-en-1-yl)acetate (5.83 g), 4-chloro-6-fluoroquinoline (3.00 g), and sodium carbonate (5.35 g) were dissolved in a mixed solvent of 1,4-dioxane (30 ml) and water (30 ml). [1,1′-bis(diphenylphosphino)ferrocene]dichloropalladium(II) (Pd(dppf)Cl₂) (675 mg) was added to the solution, which was then stirred at 95° C. overnight. The reaction mixture was concentrated and then ethyl acetate was added thereto. The mixture was washed with distilled water, dried over anhydrous magnesium sulfate and then concentrated under reduced pressure to obtain a yellow residue. The residue was purified by silica gel column chromatography (eluent: n-hexane/ethyl acetate=1/1, v/v) to give 4.40 g of the titled compound as a white solid. (Yield: 82.4%)

¹H-NMR (CDCl₃) δ 8.79 (d, 1H), 8.10 (t, 1H), 7.61 (d, 1H), 7.51 (t, 1H), 7.18 (d, 1H), 5.81 (s, 1H), 4.18 (q, 2H), 2.51-2.28 (m, 7H), 2.02 (m, 2H), 1.58 (m, 1H), 1.28 (t, 3H)

### Step 2: ethyl 2-(4-(6-fluoroquinolin-4-yl)cyclohexyl)acetate

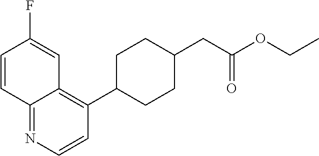 [SMILES: CCOC(=O)CC1CCC(c2ccnc3ccc(F)cc23)CC1]

A solution of ethyl 2-(4-(6-fluoroquinolin-4-yl) cyclohex-3-en-1-yl)acetate (4.40 g) prepared in Step 1 and 10% Pd/C (440 mg), and acetic acid (0.16 ml) in methanol (30 ml) was stirred for 12 hours under a hydrogen atmosphere. The reaction mixture was dried and then filtered. The resulting residue was purified by silica gel column chromatography (eluent: n-hexane/ethyl acetate=1/1, v/v) to give 4.17 g of the titled compound as a white solid. (Yield: 94.2%)

¹H-NMR (CDCl₃) δ 8.80 (s, 1H), 8.11 (t, 1H), 7.65 (d, 1H), 7.46 (t, 1H), 7.30 (d, 1H), 4.16 (q, 2H), 3.21-3.06 (m, 1H), 2.49 (s, 1H), 2.30 (d, 1H), 2.04-1.72 (m, 7H), 1.62 (q, 1H), 1.37-1.24 (m, 4H)

### Step 3: 2-(4-(6-fluoroquinolin-4-yl)cyclohexyl)ethan-1-ol

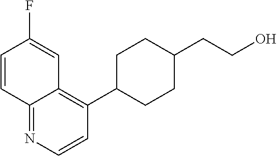 [SMILES: OCCC1CCC(c2ccnc3ccc(F)cc23)CC1]

A solution of ethyl 2-(4-(6-fluoroquinolin-4-yl)cyclohexyl)acetate (2.27 g) prepared in Step 2 in tetrahydrofuran (24 ml) was stirred at 0° C. for 10 minutes and then lithium aluminium hydride (355 mg) was slowly added thereto. The reaction mixture was stirred at room temperature for 6 hours. The reaction was quenched by adding water to the reaction mixture, which was then extracted with ethyl acetate. The organic layer was washed with brine, dried over anhydrous magnesium sulfate, and then filtered. The resulting filtrate was concentrated under reduced pressure. Two stereoisomers confirmed by TLC were collected and then purified by silica gel column chromatography (n-hexane/ethyl acetate/=1/2, v/v) to give 1.53 g of the titled compound as a white solid. (Yield: 73.2%)

¹H-NMR (DMSO-d₆) δ 8.77 (d, 1H), 8.06 (t, 1H), 7.89 (d, 1H), 7.62 (t, 1H), 7.35 (d, 1H), 4.63 (t, 1/2 H), 4.39 (t, 1/2 H), 3.47 (q, 2H), 3.20 (t, 1/2 H), 3.12 (t, 1/2 H), 1.87-1.72 (m, 4H), 1.50-1.36 (m, 4H), 1.21-1.14 (q, 2H)

### Step 4: 2-((cis)-4-(6-fluoroquinolin-4-yl)cyclohexyl)acetaldehyde

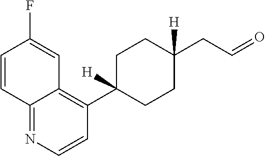 [SMILES: O=CC[C@H]1CC[C@@H](c2ccnc3ccc(F)cc32)CC1]

A solution of 2-(4-(6-fluoroquinolin-4-yl)cyclohexyl)ethan-1-ol (15.14 g) prepared in Step 3 in dichloromethane (185 ml) was stirred at 0° C. for 30 minutes and then Dess-Martin oxidizing agent (35.2 g) was slowly added thereto. The reaction mixture was stirred at room temperature for 5 hours. The reaction was quenched by adding water to the reaction mixture, which was then extracted with ethyl acetate. The organic layer was washed with brine, dried over anhydrous magnesium sulfate, and then filtered. The resulting filtrate was concentrated under reduced pressure. The lower material of the two stereoisomers confirmed by TLC in the resulting residue was purified by silica gel column chromatography (n-hexane/ethyl acetate/=3/1, v/v) to prepare 8.72 g of the titled compound as a white solid. (Yield: 57.9%)

¹H-NMR (CDCl₃) δ 9.82 (s, 1H), 8.81 (d, 1H), 8.12 (t, 1H), 7.64 (d, 1H), 7.48 (t, 1H), 7.31 (d, 1H), 3.22 (t, 1H), 2.61 (s, 3H), 1.93-1.67 (m, 8H)

### Step 5: (S)-N-(2-((cis)-4-(6-fluoroquinolin-4-yl)cyclohexyl)ethylidene)-2-methylpropan-2-sulfinamide

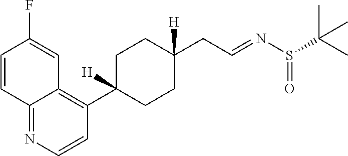 [SMILES: CC(C)(C)[S@](=O)/N=C/C[C@H]1CC[C@@H](c2ccnc3ccc(F)cc32)CC1]

A solution of 2-((cis)-4-(6-fluoroquinolin-4-yl)cyclohexyl)acetaldehyde (4.50 g) prepared in Step 4 and (S)-(-)-2-methyl-2-propanesulfinamide (4.02 g) in dichloromethane (55 ml) was stirred at 0° C. for 10 minutes and then titanium(IV) isopropoxide (9.82 ml) was slowly added thereto. The reaction mixture was stirred at room temperature for 8 hours. The reaction was quenched by adding water to the reaction mixture, which was then extracted with ethyl acetate. The organic layer was washed with brine, dried over anhydrous magnesium sulfate, and then filtered. The resulting filtrate was concentrated under reduced pressure. The resulting residue was purified by silica gel column chromatography (n-hexane/ethyl acetate/=1/1, v/v) to give 5.22 g of the titled compound as a white solid. (Yield: 84.0%)

¹H-NMR (CDCl₃) δ 8.82 (1H, d), 8.14-8.10 (m, 2H), 7.64 (d, 1H), 7.46 (t, 1H), 7.33 (d, 1H), 3.22 (t, 1H), 2.72 (t, 2H), 2.04 (s, 1H), 1.95-1.68 (m, 8H), 1.21 (s, 9H)

### Step 6: (R)-1-((cis)-4-(6-fluoroquinolin-4-yl)cyclohexyl)propan-2-amine hydrochloride

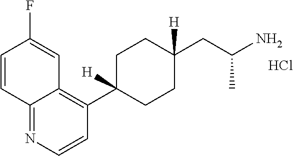 [SMILES: C[C@@H](N)C[C@H]1CC[C@@H](c2ccnc3ccc(F)cc32)CC1 ; Cl]

A solution of (S)-N-(2-((cis)-4-(6-fluoroquinolin-4-yl)cyclohexyl)ethylidene)-2-methylpropan-2-sulfinamide (2.54 g) prepared in Step 5 in dichloromethane (23 ml) was stirred at 0° C. for 10 minutes and then a solution of methylmagnesium bromide in diethyl ether (3.0 M, 4.6 ml) was slowly added thereto. The reaction mixture was stirred at room temperature for 2 hours. The reaction was quenched by adding a saturated ammonium chloride solution to the reaction mixture, which was then extracted with ethyl acetate. The organic layer was washed with brine, dried over anhydrous magnesium sulfate, and then filtered. The resulting filtrate was concentrated under reduced pressure. The resulting material was dissolved in ethyl acetate (25 ml) and then a solution of hydrochloric acid in 1,4-dioxane (4N, 2 ml) was slowly added thereto. The reaction mixture was stirred at room temperature for 8 hours. The resulting solid was filtered under reduced pressure and then washed with ethyl acetate to give 1.67 g of the titled compound as a white solid. (Yield: 86.0%)

¹H-NMR (DMSO-d₆) δ 9.23 (d, 1H), 8.56 (t, 1H), 8.42 (d, 1H), 8.27 (s, 2H), 8.08 (t, 2H), 3.63 (s, 1H), 3.19 (s, 1H), 2.11 (m, 11H), 1.26 (d, 3H)

### Preparation 2. (R)-1-((cis)-4-(6-fluoroquinolin-4-yl)cyclohexyl)butan-2-amine hydrochloride

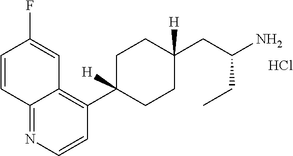 [SMILES: CC[C@@H](N)C[C@H]1CC[C@@H](c2ccnc3ccc(F)cc32)CC1 ; Cl]

The titled compound (276 mg) was prepared as a white solid in accordance with the same procedures as in Preparation 1, using a solution of ethylmagnesium bromide in tetrahydrofuran (2.0 M, 1.3 ml) instead of the solution of methylmagnesium bromide in diethyl ether. (Yield: 86.0%)

¹H-NMR (DMSO-d₆) δ 9.21 (d, 1H), 8.57 (q, 1H), 8.40 (d, 1H), 8.26 (s, 2H), 8.08 (q, 2H), 3.61 (s, 1H), 3.01 (s, 1H), 2.00-1.62 (m, 13H), 0.95 (t, 3H)

### Preparation 3. (S)-1-cyclopropyl-2-((cis)-4-(6-fluoroquinolin-4-yl)cyclohexyl)ethan-1-amine hydrochloride

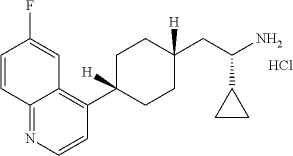 [SMILES: N[C@@H](C[C@H]1CC[C@@H](c2ccnc3ccc(F)cc32)CC1)C1CC1 ; Cl]

The titled compound (298 mg) was prepared as a white solid in accordance with the same procedures as in Preparation 1, using a solution of cyclopropylmagnesium bromide in tetrahydrofuran (1.0 M, 1.3 ml) instead of the solution of methylmagnesium bromide in diethyl ether. (Yield: 71.4%)

¹H-NMR (DMSO-d₆) δ 9.22 (d, 1H), 8.58 (q, 1H), 8.43 (d, 1H), 8.30 (s, 2H), 8.09 (t, 2H), 3.64 (s, 1H), 2.43 (s, 1H), 2.17 (s, 2H), 1.99-1.68 (m, 9H), 0.94 (s, 1H), 0.63-0.37 (m, 4H)

### Preparation 4. (R)-1-((trans)-4-(6-fluoroquinolin-4-yl)cyclohexyl)propan-2-amine hydrochloride

### Step 1: 2-((trans)-4-(6-fluoroquinolin-4-yl)cyclohexyl)acetaldehyde

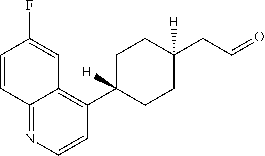 [SMILES: O=CC[C@H]1CC[C@H](c2ccnc3ccc(F)cc32)CC1]

A solution of 2-(4-(6-fluoroquinolin-4-yl)cyclohexyl)ethan-1-ol (15.14 g) prepared in Step 3 of Preparation 1 in dichloromethane (185 ml) was stirred at 0° C. for 30 minutes and then Dess-Martin oxidizing agent (35.2 g) was slowly added thereto. The reaction mixture was stirred at room temperature for 5 hours. The reaction was quenched by adding water to the reaction mixture, which was then extracted with ethyl acetate. The organic layer was washed with brine, dried over anhydrous magnesium sulfate, and then filtered. The resulting filtrate was concentrated under reduced pressure. The upper material of the two stereoisomers confirmed by TLC in the resulting residue was purified by silica gel column chromatography (n-hexane/ethyl acetate/=3/1, v/v) to prepare 5.48 g of the titled compound as a white solid. (Yield: 36.5%)

¹H-NMR (CDCl₃) δ 9.84 (s, 1H), 8.83 (d, 1H), 8.12 (dd, 1H), 7.67 (dd, 1H), 7.49 (dd, 1H), 7.30 (d, 1H), 3.19-3.13 (dt, 1H), 2.46 (dd, 2H), 2.11-2.00 (m, 5H), 1.71-1.61 (m, 2H), 1.41-1.31 (m, 2H)

### Step 2: (S)-N-(2-((trans)-4-(6-fluoroquinolin-4-yl)cyclohexyl)ethylidene)-2-methylpropan-2-sulfinamide

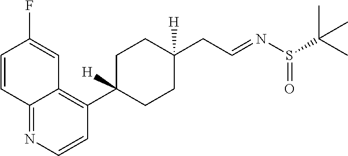 [SMILES: CC(C)(C)[S@](=O)/N=C/C[C@H]1CC[C@H](c2ccnc3ccc(F)cc32)CC1]

A solution of 2-((trans)-4-(6-fluoroquinolin-4-yl)cyclohexyl)acetaldehyde (3.00 g) prepared in Step 1 and (S)-(-)-2-methyl-2-propanesulfinamide (1.61 g) in tetrahydrofuran (40 ml) was stirred at 0° C. for 10 minutes and then titanium(IV) isopropoxide (6.55 ml) was slowly added thereto. The reaction mixture was stirred at room temperature for 8 hours. The reaction was quenched by adding water to the reaction mixture, which was then extracted with ethyl acetate. The organic layer was washed with brine, dried over anhydrous magnesium sulfate, and then filtered. The resulting filtrate was concentrated under reduced pressure. The resulting residue was purified by silica gel column chromatography (n-hexane/ethyl acetate/=1/1, v/v) to give 4.1 g of the titled compound as a white solid. (Yield: 99.0%)

¹H-NMR (CDCl₃) δ 8.82 (d, 1H), 8.13 (dd, 1H), 7.65 (dd, 1H), 7.48 (dd, 1H), 7.30 (d, 1H), 3.20-3.14 (dt, 1H), 2.56 (dd, 2H), 2.08-2.02 (m, 4H), 1.96-1.91 (m, 1H), 1.67-1.58 (m, 2H), 1.43-1.33 (m, 2H), 1.22 (s, 9H)

### Step 3: (R)-1-((trans)-4-(6-fluoroquinolin-4-yl)cyclohexyl)propan-2-amine hydrochloride

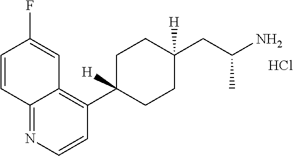 [SMILES: C[C@@H](N)C[C@H]1CC[C@H](c2ccnc3ccc(F)cc32)CC1 ; Cl]

The titled compound (1.9 g) was prepared as a white solid in accordance with the same procedures as in Step 6 of Preparation 1, using (S)-N-(2-((trans)-4-(6-fluoroquinolin-4-yl)cyclohexyl)ethylidene)-2-methylpropan-2-sulfinamide (3.2 g) prepared in Step 2 instead of (S)-N-(2-((cis)-4-(6-fluoroquinolin-4-yl)cyclohexyl)ethylidene)-2-methylpropan-2-sulfinamide. (Yield: 68.9%)

¹H-NMR (DMSO-d₆) δ 9.12 (d, 1H), 8.47 (q, 1H), 8.34 (d, 1H), 8.17 (s, 2H), 8.00 (t, 1H), 7.84 (d, 1H), 3.54 (t, 1H), 3.28 (s, 1H), 1.95-1.84 (m, 4H), 1.66-1.26 (m, 7H), 1.24 (d, 3H)

### Example 1. 4-(((R)-1-((cis)-4-(6-fluoroquinolin-4-yl)cyclohexyl)propan-2-yl)amino)quinazolin-7-carbonitrile

A solution of (R)-1-((cis)-4-(6-fluoroquinolin-4-yl)cyclohexyl)propan-2-amine hydrochloride (30 mg) prepared in Preparation 1, 4-chloroquinazolin-7-carbonitrile (19 mg), and triethylamine (23.3 μl) in ethanol (1.0 ml) was refluxed at 90° C. for 4 hours. The reaction mixture was concentrated under reduced pressure. The reaction was quenched by adding water to the reaction mixture, which was then extracted with ethyl acetate. The organic layer was washed with brine, dried over anhydrous magnesium sulfate, and then filtered. The resulting filtrate was concentrated under reduced pressure. The resulting residue was purified by silica gel column chromatography (n-hexane/ethyl acetate/=1/1, v/v) to give 12.2 mg of the titled compound as a white solid. (Yield: 33.2%)

¹H-NMR (CDCl₃) δ 8.80 (d, 1H), 8.71 (s, 1H), 8.17 (s, 1H), 8.12 (t, 1H), 7.86 (d, 1H), 7.67-7.60 (m, 2H), 7.47 (t, 1H), 7.31 (d, 1H), 5.82 (d, 1H), 4.67-4.64 (m, 1H), 3.22 (s, 1H), 2.03 (s, 1H), 1.92-1.73 (m, 10H), 1.39 (d, 3H)

### Example 2. 7-bromo-N-((R)-1-((cis)-4-(6-fluoroquinolin-4-yl)cyclohexyl)propan-2-yl)quinazolin-4-amine

A solution of (R)-1-((cis)-4-(6-fluoroquinolin-4-yl)cyclohexyl)propan-2-amine hydrochloride (120 mg) prepared in Preparation 1, 7-bromo-4-chloroquinazoline (117.6 mg), and triethylamine (155.4 μl) in ethanol (1.0 ml) was refluxed at 90° C. for 4 hours. The reaction mixture was concentrated under reduced pressure. The reaction was quenched by adding water to the reaction mixture, which was then extracted with ethyl acetate. The organic layer was washed with brine, dried over anhydrous magnesium sulfate, and then filtered. The resulting filtrate was concentrated under reduced pressure. The resulting residue was purified by silica gel column chromatography (n-hexane/ethyl acetate/=1/1, v/v) to give 113.0 mg of the titled compound as a white solid. (Yield: 61.6%)

¹H-NMR (CDCl₃) δ 8.80 (d, 1H), 8.63 (s, 1H), 8.11 (t, 1H), 8.01 (s, 1H), 7.65 (d, 1H), 7.57 (dd, 2H), 7.46 (t, 1H), 7.31 (s, 1H), 5.60 (d, 1H), 4.66-4.61 (m, 1H), 3.20 (s, 1H), 2.03 (s, 1H), 1.89-1.71 (m, 10H), 1.37 (d, 3H)

### Example 3. 7-chloro-N-((R)-1-((cis)-4-(6-fluoroquinolin-4-yl)cyclohexyl)propan-2-yl)quinazolin-4-amine

A solution of (R)-1-((cis)-4-(6-fluoroquinolin-4-yl)cyclohexyl)propan-2-amine hydrochloride (57.5 mg) prepared in Preparation 1, 4,7-dichloroquinazoline (50 mg), and triethylamine (23.3 μl) in ethanol (1.0 ml) was refluxed at 90° C. for 4 hours. The reaction mixture was concentrated under reduced pressure. The reaction was quenched by adding water to the reaction mixture, which was then extracted with ethyl acetate. The organic layer was washed with brine, dried over anhydrous magnesium sulfate, and then filtered. The resulting filtrate was concentrated under reduced pressure. The resulting residue was purified by silica gel column chromatography (n-hexane/ethyl acetate/=1/1, v/v) to give 52.2 mg of the titled compound as a white solid. (Yield: 46.3%)

¹H-NMR (CDCl₃) δ 8.80 (d, 1H), 8.64 (s, 1H), 8.12-8.10 (m, 1H), 7.84 (s, 1H), 7.67-7.63 (m, 2H), 7.48-7.42 (m, 2H), 7.31 (s, 1H), 5.43 (m, 1H), 4.64-4.62 (m, 1H), 3.21 (m, 1H) 1.86-1.44 (m, 11H), 1.38 (d, 3H)

### Example 4. 7-chloro-N-((R)-1-((trans)-4-(6-fluoroquinolin-4-yl)cyclohexyl)propan-2-yl)quinazolin-4-amine

A solution of (R)-1-((trans)-4-(6-fluoroquinolin-4-yl)cyclohexyl)propan-2-amine hydrochloride (130 mg) prepared in Preparation 4, 4,7-dichloroquinazoline (180.7 mg), and triethylamine (190 μl) in ethanol (1.0 ml) was refluxed at 90° C. for 4 hours. The reaction mixture was concentrated under reduced pressure. The reaction was quenched by adding water to the reaction mixture, which was then extracted with ethyl acetate. The organic layer was washed with brine, dried over anhydrous magnesium sulfate, and then filtered. The resulting filtrate was concentrated under reduced pressure. The resulting residue was purified by silica gel column chromatography (n-hexane/ethyl acetate/=1/1, v/v) to give 45.2 mg of the titled compound as a white solid. (Yield: 34.8%)

¹H-NMR (CDCl₃) δ 8.78 (d, 1H), 8.65 (s, 1H), 8.12-8.08 (m, 1H), 7.84 (s, 1H), 7.66-7.63 (m, 2H), 7.48-7.42 (m, 2H), 7.24 (d, 1H), 5.44 (m, 1H), 4.72 (m, 1H), 3.18-3.12 (m, 1H), 2.11 (d, 1H), 2.05-2.00 (m, 3H), 1.70-1.53 (m, 7H), 1.38 (d, 3H)

### Example 5. 7-bromo-4-(((R)-1-((trans)-4-(6-fluoroquinolin-4-yl)cyclohexyl)propan-2-yl)amino)-2H-chromen-2-one

A solution of (R)-1-((trans)-4-(6-fluoroquinolin-4-yl)cyclohexyl)propan-2-amine hydrochloride (30 mg) prepared in Preparation 4, 7-bromo-4-chloro-2H-chromen-2-one (25.9 mg), and triethylamine (40.0 μl) in ethanol (1.0 ml) was refluxed at 90° C. for 4 hours. The reaction mixture was concentrated under reduced pressure. The reaction was quenched by adding water to the reaction mixture, which was then extracted with ethyl acetate. The organic layer was washed with brine, dried over anhydrous magnesium sulfate, and then filtered. The resulting filtrate was concentrated under reduced pressure. The resulting residue was purified by silica gel column chromatography (n-hexane/ethyl acetate/=1/1, v/v) to give 23.2 mg of the titled compound as a white solid. (Yield: 43.8%)

¹H-NMR (CDCl₃) δ: 8.78 (d, 1H), 8.12-8.10 (m, 1H), 7.65 (dd, 1H), 7.50-7.41 (m, 4H), 7.27 (s, 1H), 5.35 (s, 1H), 5.15 (d, 1H), 3.78-3.72 (m, 1H), 3.18-3.10 (m, 1H), 2.10-1.89 (m, H), 1.86-1.44 (m, 5H), 1.40-1.18 (m, 9H)

### Example 6. 7-chloro-4-(((R)-1-((trans)-4-(6-fluoroquinolin-4-yl)cyclohexyl)propan-2-yl)amino)-2H-chromen-2-one

A solution of (R)-1-((trans)-4-(6-fluoroquinolin-4-yl)cyclohexyl)propan-2-amine hydrochloride (30 mg) prepared in Preparation 4, 4,7-dichloro-2H-chromen-2-one (45.1 mg), and triethylamine (40.0 μl) in ethanol (1.0 ml) was refluxed at 90° C. for 4 hours. The reaction mixture was concentrated under reduced pressure. The reaction was quenched by adding water to the reaction mixture, which was then extracted with ethyl acetate. The organic layer was washed with brine, dried over anhydrous magnesium sulfate, and then filtered. The resulting filtrate was concentrated under reduced pressure. The resulting residue was purified by silica gel column chromatography (n-hexane/ethyl acetate/=1/1, v/v) to give 11.0 mg of the titled compound as a white solid. (Yield: 22.6%)

¹H-NMR (CDCl₃) δ 8.78 (d, 1H), 8.13-8.09 (m, 1H), 7.66-7.63 (d, 1H), 7.50-7.40 (m, 2H), 7.35 (s, 1H), 7.26-7.24 (m, 2H), 5.35 (s, 1H), 4.96 (d, 1H), 3.82-3.78 (m, 1H), 3.18-3.12 (m, 1H), 2.08-2.04 (m, 4H), 1.75 (m, 1H), 1.63-1.56 (m, 4H), 1.38 (d, 3H)

### Example 7. 6-bromo-4-(((R)-1-((trans)-4-(6-fluoroquinolin-4-yl)cyclohexyl)propan-2-yl)amino)-2H-chromen-2-one

A solution of (R)-1-((trans)-4-(6-fluoroquinolin-4-yl)cyclohexyl)propan-2-amine hydrochloride (80.8 mg) prepared in Preparation 4, 6-bromo-4-chloro-2H-chromen-2-one (50 mg), and triethylamine (23.3 μl) in ethanol (1.0 ml) was refluxed at 90° C. for 4 hours. The reaction mixture was concentrated under reduced pressure. The reaction was quenched by adding water to the reaction mixture, which was then extracted with ethyl acetate. The organic layer was washed with brine, dried over anhydrous magnesium sulfate, and then filtered. The resulting filtrate was concentrated under reduced pressure. The resulting residue was purified by silica gel column chromatography (n-hexane/ethyl acetate/=1/1, v/v) to give 54.3 mg of the titled compound as a white solid. (Yield: 55.3%)

¹H-NMR (CDCl₃) δ 8.81 (d, 1H), 8.26 (t, 1H), 7.71-7.60 (m, 3H), 7.52 (t, 1H), 7.34 (d, 1H), 7.23 (t, 1H), 5.38 (s, 1H), 5.04 (d, 1H), 3.83-3.78 (m, 1H), 3.19 (t, 1H), 2.12-1.98 (m, 4H), 1.77 (t, 1H), 1.64-1.55 (m, 4H), 1.38-1.22 (m, 5H)

### Example 8. 7-chloro-4-(((R)-1-((cis)-4-(6-fluoroquinolin-4-yl)cyclohexyl)propan-2-yl)amino)-2H-chromen-2-one

A solution of (R)-1-((cis)-4-(6-fluoroquinolin-4-yl)cyclohexyl)propan-2-amine hydrochloride (30 mg) prepared in Preparation 1, 4,7-dichloro-2H-chromen-2-one (45.1 mg), and triethylamine (40.0 μl) in ethanol (1.0 ml) was refluxed at 90° C. for 4 hours. The reaction mixture was concentrated under reduced pressure. The reaction was quenched by adding water to the reaction mixture, which was then extracted with ethyl acetate. The organic layer was washed with brine, dried over anhydrous magnesium sulfate, and then filtered. The resulting filtrate was concentrated under reduced pressure. The resulting residue was purified by silica gel column chromatography (n-hexane/ethyl acetate/=1/1, v/v) to give 5.5 mg of the titled compound as a white solid. (Yield: 11.3%)

¹H-NMR (CDCl₃) δ 8.80 (d, 1H), 8.13-8.10 (m, 1H), 7.66-7.63 (d, 1H), 7.50-7.45 (m, 1H), 7.39-7.31 (m, 3H), 7.26-7.24 (m, 1H), 5.35 (s, 1H), 4.88 (d, 1H), 3.74-3.70 (m, 1H), 3.23-3.20 (m, 1H), 2.04-2.00 (m, 1H), 1.88-1.64 (m, 10H), 1.36 (d, 3H)

### Example 9. 7-bromo-4-(((R)-1-((cis)-4-(6-fluoroquinolin-4-yl)cyclohexyl)propan-2-yl)amino)-2H-chromen-2-one

A solution of (R)-1-((cis)-4-(6-fluoroquinolin-4-yl)cyclohexyl)propan-2-amine hydrochloride (30 mg) prepared in Preparation 1, 7-bromo-4-chloro-2H-chromen-2-one (25.9 mg), and triethylamine (40.0 μl) in ethanol (1.0 ml) was refluxed at 90° C. for 4 hours. The reaction mixture was concentrated under reduced pressure. The reaction was quenched by adding water to the reaction mixture, which was then extracted with ethyl acetate. The organic layer was washed with brine, dried over anhydrous magnesium sulfate, and then filtered. The resulting filtrate was concentrated under reduced pressure. The resulting residue was purified by silica gel column chromatography (n-hexane/ethyl acetate/=1/1, v/v) to give 19.5 mg of the titled compound as a white solid. (Yield: 36.0%)

¹H-NMR (CDCl₃) δ 8.80 (d, 1H), 8.13-8.10 (dd, 1H), 7.64 (dd, 1H), 7.50-7.44 (m, 2H), 7.38 (s, 2H), 7.31 (d, 1H), 5.35 (s, 1H), 5.10 (d, 1H), 3.74-3.68 (m, 1H), 3.22-3.20 (m, 1H), 2.07-1.97 (m, 1H), 1.92-1.65 (m, 10H), 1.35 (d, 3H), 1.25 (s, 2H)

### Example 10. 7-chloro-3-fluoro-4-(((R)-1-((trans)-4-(6-fluoroquinolin-4-yl)cyclohexyl)propan-2-yl)amino)-2H-chromen-2-one

7-Chloro-4-(((R)-1-((trans)-4-(6-fluoroquinolin-4-yl)cyclohexyl)propan-2-yl)amino)-2H-chromen-2-one hydrochloride (100 mg) prepared in Example 6 was dissolved in a mixed solvent of methanol (1.0 ml) and acetonitrile (1.0 ml). The reaction mixture was stirred at −10° C. for 10 minutes and then Selectfluor (83.4 mg) was slowly added thereto. The reaction mixture was stirred at −10° C. for 10 minutes and then stirred at room temperature for 1 hour. The reaction mixture was concentrated under reduced pressure. The reaction was quenched by adding water to the reaction mixture, which was then extracted with ethyl acetate. The organic layer was washed with brine, dried over anhydrous magnesium sulfate, and then filtered. The resulting filtrate was concentrated under reduced pressure. The resulting residue was purified by silica gel column chromatography (n-hexane/ethyl acetate/=1/1, v/v) to give 5.2 mg of the titled compound as a white solid. (Yield: 84.0%)

¹H-NMR (CDCl₃) δ 8.79 (d, 1H), 8.11 (t, 1H), 7.05 (t, 1H), 7.47-7.26 (m, 5H), 4.35 (d, 2H), 3.74 (m, 1H), 3.20-3.09 (m, 1H), 2.11-1.96 (m, 4H), 1.88-1.82 (m, 1H), 1.75-1.49 (m, 4H), 1.46-1.32 (m, 5H)

### Example 11. 7-bromo-3-fluoro-4-(((R)-1-((cis)-4-(6-fluoroquinolin-4-yl)cyclohexyl)propan-2-yl)amino)-2H-chromen-2-one

7-Bromo-4-(((R)-1-((cis)-4-(6-fluoroquinolin-4-yl)cyclohexyl)propan-2-yl)amino)-2H-chromen-2-one (68 mg) prepared in Example 9 was dissolved in a mixed solvent of methanol (1.0 ml) and acetonitrile (1.0 ml). The reaction mixture was stirred at −10° C. for minutes and then Selectfluor (77.7 mg) was slowly added thereto. The reaction mixture was stirred at −10° C. for 10 minutes and then stirred at room temperature for 1 hour. The reaction mixture was concentrated under reduced pressure. The reaction was quenched by adding water to the reaction mixture, which was then extracted with ethyl acetate. The organic layer was washed with brine, dried over anhydrous magnesium sulfate, and then filtered. The resulting filtrate was concentrated under reduced pressure. The resulting residue was purified by silica gel column chromatography (n-hexane/ethyl acetate/=1/1, v/v) to give 61.0 mg of the titled compound as a white solid. (Yield: 86.4%)

¹H-NMR (CDCl₃) δ 8.80 (d, 1H), 8.11 (t, 1H), 7.65 (d, 1H), 7.51-7.26 (m, 4H), 6.98 (s, 1H), 4.48 (d, 1H), 4.26-4.22 (m, 1H), 3.27-3.16 (m, 1H), 2.27 (s, 1H), 2.05 (s, 2H), 1.91-1.64 (m, 8H), 1.38 (d, 3H)

### Example 12. 7-bromo-3-fluoro-4-(((R)-1-((trans)-4-(6-fluoroquinolin-4-yl)cyclohexyl)propan-2-yl)amino)-2H-chromen-2-one

7-Bromo-4-(((R)-1-((trans)-4-(6-fluoroquinolin-4-yl)cyclohexyl)propan-2-yl)amino)-2H-chromen-2-one (100 mg) prepared in Example 5 was dissolved in a mixed solvent of methanol (1.0 ml) and acetonitrile (1.0 ml). The reaction mixture was stirred at −10° C. for 10 minutes and then Selectfluor (83.4 mg) was slowly added thereto. The reaction mixture was stirred at −10° C. for 10 minutes and then stirred at room temperature for 1 hour. The reaction mixture was concentrated under reduced pressure. The reaction was quenched by adding water to the reaction mixture, which was then extracted with ethyl acetate. The organic layer was washed with brine, dried over anhydrous magnesium sulfate, and then filtered. The resulting filtrate was concentrated under reduced pressure. The resulting residue was purified by silica gel column chromatography (n-hexane/ethyl acetate/=1/1, v/v) to give 16.0 mg of the titled compound as a white solid. (Yield: 58.9%)

¹H-NMR (CDCl₃) δ 8.79 (d, 1H), 8.13-8.09 (m, 1H), 7.65 (d, 1H), 7.52 (s, 1H), 7.49-7.44 (m, 2H), 7.36 (d, 2H), 4.42-4.33 (m, 2H), 3.15 (t, 1H), 2.04-1.99 (m, 4H), 1.72-1.51 (m, 7H), 1.36 (d, 3H)

### Example 13. 6-chloro-N-((R)-1-((cis)-4-(6-fluoroquinolin-4-yl)cyclohexyl)propan-2-yl)benzo[d]thiazol-2-amine

(R)-1-((cis)-4-(6-fluoroquinolin-4-yl)cyclohexyl)propan-2-amine hydrochloride (150 mg) prepared in Preparation 1 and 2,6-dichloro-1,3-benzothiazole (22 mg) were dissolved in N-methyl-2-pyrrolidone (1.0 ml) and then N,N-diisopropylethylamine (55 μl) was slowly added thereto. The reaction mixture was stirred in a microwave reactor (180° C., 1200 W) for 2 hours and then cooled to room temperature. The reaction was quenched by adding water to the reaction mixture, which was then extracted with ethyl acetate. The organic layer was washed with brine, dried over anhydrous magnesium sulfate, and then filtered. The resulting filtrate was concentrated under reduced pressure. The resulting residue was purified by silica gel column chromatography (n-hexane/ethyl acetate/=1/1, v/v) to give 10.7 mg of the titled compound as a white solid. (Yield: 22.5%)

¹H-NMR (CDCl₃) δ 8.78 (d, 1H), 8.13-8.10 (m, 1H), 7.64 (d, 1H), 7.54 (s, 1H), 7.48-7.39 (m, 2H), 7.28-7.23 (m, 2H), 5.48 (m, 1H), 3.84 (m, 1H), 3.12 (m, 1H), 2.04 (s, 2H), 1.80-1.49 (m, 8H), 1.36 (d, 3H)

### Example 14. N-((R)-1-((cis)-4-(6-fluoroquinolin-4-yl)cyclohexyl)propan-2-yl)-6-methoxybenzo[d]thiazol-2-amine

The titled compound (5.2 mg) was prepared in accordance with the same procedures as in Example 13, using 2-chloro-6-methoxy-1,3-benzothiazole (21 mg) instead of 2,6-dichloro-1,3-benzothiazole. (Yield: 11.0%)

¹H-NMR (CDCl₃) δ 8.78 (d, 1H), 8.13-8.09 (m, 1H), 7.65 (d, 1H), 7.48-7.41 (m, 2H), 7.30 (d, 1H), 7.13 (s, 1H), 6.88 (d, 1H), 5.04 (m, 1H), 3.82-3.78 (m, 4H), 3.21-3.19 (m, 1H), 2.08-2.04 (m, 1H), 1.81-1.51 (m, 10H), 1.38 (d, 3H)

### Example 15. N-((R)-1-((cis)-4-(6-fluoroquinolin-4-yl)cyclohexyl)butan-2-yl)-6-methoxybenzo[d]thiazol-2-amine

(R)-1-((cis)-4-(6-fluoroquinolin-4-yl)cyclohexyl)butan-2-amine hydrochloride (35.5 mg) prepared in Preparation 2 and 2-chloro-6-methoxy-1,3-benzothiazole (30 mg) were dissolved in N-methyl-2-pyrrolidone (1.0 ml) and then N,N-diisopropylethylamine (78.5 μl) was slowly added thereto. The reaction mixture was stirred in a microwave reactor (180° C., 1200 W) for 2 hours and then cooled to room temperature. The reaction was quenched by adding water to the reaction mixture, which was then extracted with ethyl acetate. The organic layer was washed with brine, dried over anhydrous magnesium sulfate, and then filtered. The resulting filtrate was concentrated under reduced pressure. The resulting residue was purified by silica gel column chromatography (n-hexane/ethyl acetate/=1/1, v/v) to give 22 mg of the titled compound as a white solid. (Yield: 31.6%)

¹H-NMR (CDCl₃) δ 8.77 (d, 1H), 8.10 (t, 1H), 7.65 (d, 1H), 7.45 (t, 1H), 7.39 (d, 1H), 7.30-7.23 (m, 1H), 7.12 (s, 1H), 6.88 (d, 1H), 5.21 (s, 1H), 3.81 (s, 3H), 3.61 (d, 1H), 3.20-3.08 (m, 1H), 2.04 (s, 1H), 1.85-1.53 (m, 10H), 1.34-1.24 (m, 2H), 1.01 (t, 3H)

### Example 16. N-((S)-1-cyclopropyl-2-((cis)-4-(6-fluoroquinolin-4-yl)cyclohexyl)ethyl)-6-methoxybenzo[d]thiazol-2-amine

(S)-1-cyclopropyl-2-((cis)-4-(6-fluoroquinolin-4-yl)cyclohexyl)ethan-1-amine hydrochloride (36.7 mg) prepared in Preparation 3 and 2-chloro-6-methoxy-1,3-benzothiazole (30 mg) were dissolved in N-methyl-2-pyrrolidone (1.0 ml) and then N,N′-diisopropylethylamine (78.5 μl) was slowly added thereto. The reaction mixture was stirred in a microwave reactor (180° C., 1200 W) for 2 hours and then cooled to room temperature. The reaction was quenched by adding water to the reaction mixture, which was then extracted with ethyl acetate. The organic layer was washed with brine, dried over anhydrous magnesium sulfate, and then filtered. The resulting filtrate was concentrated under reduced pressure. The resulting residue was purified by silica gel column chromatography (n-hexane/ethyl acetate/=1/1, v/v) to give 27.3 mg of the titled compound as a white solid. (Yield: 38.2%)

¹H-NMR (CDCl₃) δ 8.77 (d, 1H), 8.10 (t, 1H), 7.65 (d, 1H), 7.46 (t, 1H), 7.40 (d, 1H), 7.39-7.23 (m, 1H), 7.12 (s, 1H), 6.88 (d, 1H), 5.19 (s, 1H), 3.81 (s, 3H), 3.33-3.09 (m, 2H), 2.04-1.53 (m, 10H), 0.97 (t, 1H), 0.61-0.34 (m, 4H)

### Example 17. 4,6-difluoro-N-((R)-1-((cis)-4-(6-fluoroquinolin-4-yl)cyclohexyl)propan-2-yl)benzo[d]thiazol-2-amine

(R)-1-((cis)-4-(6-fluoroquinolin-4-yl)cyclohexyl)propan-2-amine hydrochloride (34 mg) prepared in Preparation 1, 2-chloro-4,6-difluorobenzothiazole (21.3 mg), and copper(I) iodide (6 mg) were dissolved in dimethyl sulfoxide (1.0 ml) and then potassium carbonate (43 mg) was added thereto. The reaction mixture was stirred in a microwave reactor (100° C., 600 W) for 3 hours and then cooled to room temperature. The reaction was quenched by adding water to the reaction mixture, which was then extracted with ethyl acetate. The organic layer was washed with ammonia solution and brine, dried over anhydrous magnesium sulfate, and then filtered. The resulting filtrate was concentrated under reduced pressure. The resulting residue was purified by silica gel column chromatography (n-hexane/ethyl acetate/=1/2, v/v) to give 25 mg of the titled compound as a white solid. (Yield: 52.4%)

¹H-NMR (CDCl₃) δ 8.79 (s, 1H), 8.12 (t, 1H), 7.64 (d, 1H), 7.46 (t, 1H), 7.27 (d, 1H), 7.11 (d, 1H), 6.83 (t, 1H), 5.81 (d, 1H), 3.85-3.72 (m, 1H), 3.25-3.10 (m, 1H), 2.05 (s, 2H), 1.86-1.58 (m, 9H), 1.36 (d, 3H)

### Example 18. 7-chloro-N-((R)-1-((cis)-4-(6-fluoroquinolin-4-yl)cyclohexyl)propan-2-yl)benzo[d]thiazol-2-amine

The titled compound (18.4 mg) was prepared in accordance with the same procedures as in Example 17, using 2,7-dichlorobenzo[d]thiazole (21.3 mg) instead of 2-chloro-4,6-difluorobenzothiazole. (Yield: 38.7%)

¹H-NMR (CDCl₃) δ 8.78 (s, 1H), 8.13-8.10 (m, 1H), 7.64 (d, 1H), 7.45 (t, 1H), 7.39 (d, 1H), 7.28-7.21 (m, 2H), 7.06 (d, 1H), 5.72 (s, 1H), 3.84-3.83 (m, 1H), 3.18 (t, 1H), 2.04 (s, 2H), 1.80-1.66 (m, 9H), 1.38 (d, 3H)

### Example 19. 6-ethoxy-N-((R)-1-((cis)-4-(6-fluoroquinolin-4-yl)cyclohexyl)propan-2-yl)benzo[d]thiazol-2-amine

(R)-1-((cis)-4-(6-fluoroquinolin-4-yl)cyclohexyl)propan-2-amine hydrochloride (20 mg) prepared in Preparation 1, 2-chloro-6-ethoxy-1,3-benzothiazole (23 mg), and copper(I) iodide (4 mg) were dissolved in dimethyl sulfoxide (1.0 ml) and then potassium carbonate (48 mg) was added thereto. The reaction mixture was stirred in a microwave reactor (100° C., 600 W) for 3 hours and then cooled to room temperature. The reaction was quenched by adding water to the reaction mixture, which was then extracted with ethyl acetate. The organic layer was washed with ammonia solution and brine, dried over anhydrous magnesium sulfate, and then filtered. The resulting filtrate was concentrated under reduced pressure. The resulting residue was purified by silica gel column chromatography (n-hexane/ethyl acetate/=1/2, v/v) to give 2.3 mg of the titled compound. (Yield: 7.1%)

¹H-NMR (CDCl₃) δ 8.79 (d, 1H), 8.13-8.10 (m, 1H), 7.65 (d, 1H), 7.47 (t, 1H), 7.43 (d, 1H), 7.31 (d, 1H), 7.13 (s, 1H), 6.88 (d, 1H), 5.00 (m, 1H), 4.03 (q, 2H), 3.83 (m, 1H), 3.21 (m, 1H), 2.04 (br, 1H), 1.81-1.63 (m, 9H), 1.41 (t, 3H), 1.36 (d, 3H)

### Example 20. 6-ethoxy-N-((R)-1-((cis)-4-(6-fluoroquinolin-4-yl)cyclohexyl)butan-2-yl)benzo[d]thiazol-2-amine

(R)-1-((cis)-4-(6-fluoroquinolin-4-yl)cyclohexyl)butan-2-amine hydrochloride (30 mg) prepared in Preparation 2, 2-chloro-6-ethoxy-1,3-benzothiazole (18 mg), copper(I) iodide (6 mg) were dissolved in dimethyl sulfoxide (1.0 ml) and then potassium carbonate (60 mg) was added thereto. The reaction mixture was stirred in a microwave reactor (120° C., 600 W) for 2 hours and then cooled to room temperature. The reaction was quenched by adding water to the reaction mixture, which was then extracted with ethyl acetate. The organic layer was washed with ammonia solution and brine, dried over anhydrous magnesium sulfate, and then filtered. The resulting filtrate was concentrated under reduced pressure. The resulting residue was purified by silica gel column chromatography (n-hexane/ethyl acetate/=1/2, v/v) to give 7.8 mg of the titled compound as a white solid. (Yield: 18.7%)

¹H-NMR (CDCl₃) δ 8.78 (d, 1H), 8.12-8.09 (m, 1H), 7.65 (d, 1H), 7.46-7.38 (m, 2H), 7.28 (d, 1H), 7.12 (s, 1H), 6.88 (d, 1H), 5.18 (m, 1H), 4.03 (d, 2H), 3.59 (s, 1H), 3.20 (s, 1H), 2.04 (s, 1H), 1.81-1.56 (m, 12H), 1.41 (t, 3H), 1.02 (t, 3H)

### Example 21. N-((R)-1-((cis)-4-(6-fluoroquinolin-4-yl)cyclohexyl)propan-2-yl)thiazolo[4,5-b]pyridin-2-amine

The titled compound (19.2 mg) was prepared in accordance with the same procedures as in Example 19, using 2-chlorothiazolo[4,5-b]pyridine (18 mg) instead of 2-chloro-6-ethoxy-1,3-benzothiazole. (Yield: 43.6%)

¹H-NMR (CDCl₃) δ 8.77 (d, 1H), 8.55 (s, 1H), 8.12-8.08 (m, 1H), 7.84 (d, 1H), 7.63 (d, 1H), 7.47-7.43 (m, 1H), 7.23 (d, 1H), 6.95 (t, 1H), 6.65 (s, 1H), 3.95 (s, 1H), 3.22 (s, 1H), 2.04-1.94 (m, 2H), 1.78-1.41 (m, 9H), 1.41 (d, 3H)

### Example 22. N-((R)-1-((cis)-4-(6-fluoroquinolin-4-yl)cyclohexyl)propan-2-yl)-5-methoxythiazolo[5,4-b]pyridin-2-amine

The titled compound (11.6 mg) was prepared in accordance with the same procedures as in Example 19, using 2-chloro-5-methoxy-thiazolo[5,4-b]pyridine (21.1 mg) instead of 2-chloro-6-ethoxy-1,3-benzothiazole. (Yield: 18.4%)

¹H-NMR (CDCl₃) δ 8.80 (d, 1H), 8.13-8.10 (m, 1H), 7.66-7.64 (m, 2H), 7.46-7.44 (m, 1H), 7.31 (d, 1H), 6.68 (d, 1H), 5.08 (m, 1H), 3.93 (s, 3H), 3.89 (m, 1H), 3.22 (m, 1H), 2.04-2.00 (m, 1H), 1.88-1.64 (m, 10H), 1.36 (d, 3H)

### Example 23. 6-chloro-N-((R)-1-((cis)-4-(6-fluoroquinolin-4-yl)cyclohexyl)propan-2-yl)thiazolo[4,5-b]pyridin-2-amine

The titled compound (2 mg) was prepared in accordance with the same procedures as in Example 19, using 2,6-dichlorothiazolo[4,5-b]pyridine (18 mg) instead of 2-chloro-6-ethoxy-1,3-benzothiazole. (Yield: 6.0%)

¹H-NMR (CDCl₃) δ 8.80 (d, 1H), 8.41-8.30 (bs, 1H), 8.12-8.09 (m, 1H), 7.83 (s, 1H), 7.65 (d, 1H), 7.49-7.60 (m, 1H), 7.29 (d, 1H), 6.32 (bs, 1H), 3.97 (m, 1H), 3.76 (m, 1H), 3.20-3.15 (m, 1H), 2.05-1.26 (m, 13H)

### Example 24. N-((R)-1-((cis)-4-(6-fluoroquinolin-4-yl)cyclohexyl)propan-2-yl)benzo[d]oxazol-2-amine

(R)-1-((cis)-4-(6-fluoroquinolin-4-yl)cyclohexyl)propan-2-amine hydrochloride (100 mg) prepared in Preparation 1 and 2-chlorobenzo[d]oxazole (71 mg) were dissolved in N,N-dimethylformamide (1.0 ml) and then 1,8-diazabicyclo[5,4,0]undec-7-ene (71 mg) was slowly added thereto. The reaction mixture was stirred in a microwave reactor (180° C., 1200 W) for 2 hours and then cooled to room temperature. The reaction was quenched by adding water to the reaction mixture, which was then extracted with ethyl acetate. The organic layer was washed with brine, dried over anhydrous magnesium sulfate, and then filtered. The resulting filtrate was concentrated under reduced pressure. The resulting residue was purified by silica gel column chromatography (n-hexane/ethyl acetate/=1/2, v/v) to give 12.1 mg of the titled compound. (Yield: 8.2%)

¹H-NMR (CDCl₃) δ 8.77 (d, 1H), 8.12-8.08 (m, 1H), 7.66-7.63 (m, 1H), 7.47 (t, 1H), 7.43 (d, 1H), 7.37 (d, 1H), 7.26-7.23 (m, 2H), 7.16 (t, 1H), 7.03 (t, 1H), 5.00 (d, 1H), 4.14-4.09 (m, 1H), 3.12 (t, 1H), 2.13-1.95 (m, 4H), 1.81 (s, 1H), 1.66-1.50 (m, 4H), 1.38-1.24 (m, 5H)

### Example 25. 6-chloro-N-((R)-1-((cis)-4-(6-fluoroquinolin-4-yl)cyclohexyl)propan-2-yl)benzo[d]oxazol-2-amine

(R)-1-((cis)-4-(6-fluoroquinolin-4-yl)cyclohexyl)propan-2-amine hydrochloride (22 mg) prepared in Preparation 1 and 2,6-dichlorobenzoxazole (15 mg) were dissolved in 1,4-dioxane (1.0 ml) and then N,N-diisopropylethylamine (40 μL) was slowly added thereto. The reaction mixture was stirred at 80° C. for 12 hours and then cooled to room temperature. The reaction was quenched by adding water to the reaction mixture, which was then extracted with ethyl acetate. The organic layer was washed with brine, dried over anhydrous magnesium sulfate, and then filtered. The resulting filtrate was concentrated under reduced pressure. The resulting residue was purified by silica gel column chromatography (n-hexane/ethyl acetate/=1/2, v/v) to give 14.7 mg of the titled compound. (Yield: 43.7%)

¹H-NMR (CDCl₃) δ 8.79 (d, 1H), 8.13-8.10 (m, 1H), 7.67-7.64 (d, 1H), 7.49-7.44 (m, 1H), 7.31 (d, 1H), 7.26-7.24 (m, 1H), 7.15 (d, 1H), 4.92 (d, 1H), 4.06-4.02 (m, 1H), 3.21-3.19 (m, 1H), 2.04-2.00 (m, 1H), 1.88-1.64 (m, 11H), 1.36 (d, 3H)

### Example 26. N-((R)-1-((cis)-4-(6-fluoroquinolin-4-yl)cyclohexyl)propan-2-yl)-6-methoxybenzo[d]oxazol-2-amine

(R)-1-((cis)-4-(6-fluoroquinolin-4-yl)cyclohexyl)propan-2-amine hydrochloride (50 mg) prepared in Preparation 1, 2-chloro-6-methoxy-1,3-benzoxazole hydrochloride (50 mg), and potassium carbonate (43 mg) were dissolved in N,N-dimethylformamide (1.0 ml) and then triethylamine (40 μL) was slowly added thereto. The reaction mixture was stirred at 80° C. for 12 hours and then cooled to room temperature. The reaction was quenched by adding water to the reaction mixture, which was then extracted with ethyl acetate. The organic layer was washed with brine, dried over anhydrous magnesium sulfate, and then filtered. The resulting filtrate was concentrated under reduced pressure. The resulting residue was purified by silica gel column chromatography (n-hexane/ethyl acetate/=1/2, v/v) to give 8.3 mg of the titled compound. (Yield: 12.4%)

¹H-NMR (CDCl₃) δ 8.79 (d, 1H), 8.13-8.09 (m, 1H), 7.65 (d, 1H), 7.46 (t, 1H), 7.31 (d, 1H), 7.25 (d, 1H), 6.86 (s, 1H) 6.76 (d, 1H), 4.74 (bs, 1H), 4.11 (t, 1H), 3.81 (s, 3H), 3.21 (s, 1H), 2.04 (s, 1H), 1.81-1.70 (m, 10H), 1.36 (d, 3H)

### Example 27. 6-chloro-N-((R)-1-((cis)-4-(6-fluoroquinolin-4-yl)cyclohexyl)butan-2-yl)benzo[d]oxazol-2-amine

(R)-1-((cis)-4-(6-fluoroquinolin-4-yl)cyclohexyl)butan-2-amine hydrochloride (70 mg) prepared in Preparation 2, 2,6-dichlorobenzoxazole (32 mg), and potassium carbonate (57 mg) were dissolved in N,N-dimethylformamide (1.0 ml) and then triethylamine (30 μL) was slowly added thereto. The reaction mixture was stirred at 80° C. for 12 hours and then cooled to room temperature. The reaction was quenched by adding water to the reaction mixture, which was then extracted with ethyl acetate. The organic layer was washed with brine, dried over anhydrous magnesium sulfate, and then filtered. The resulting filtrate was concentrated under reduced pressure. The resulting residue was purified by silica gel column chromatography (n-hexane/ethyl acetate/=1/2, v/v) to give 42 mg of the titled compound. (Yield: 44.7%)

¹H-NMR (CDCl₃) δ 8.79 (d, 1H), 8.13-8.09 (m, 1H), 7.64 (d, 1H), 7.45 (t, 1H), 7.26-7.21 (m, 3H), 7.14 (d, 1H), 5.60 (s, 1H), 3.88 (s, 1H), 3.19 (s, 1H), 2.04 (s, 1H), 1.82-1.61 (m, 12H), 1.01 (t, 3H)

### Example 28. 4-fluoro-N-((R)-1-((cis)-4-(6-fluoroquinolin-4-yl)cyclohexyl)propan-2-yl)benzo[d]oxazol-2-amine

(R)-1-((cis)-4-(6-fluoroquinolin-4-yl)cyclohexyl)propan-2-amine hydrochloride (30 mg) prepared in Preparation 1 and 2-chloro-4-fluoro-1,3-benzoxazole (16 mg) were dissolved in 1,4-dioxane (1.0 ml) and then N,N-diisopropylethylamine (50 μL) was slowly added thereto. The reaction mixture was stirred at 100° C. for 12 hours and then cooled to room temperature. The reaction was quenched by adding water to the reaction mixture, which was then extracted with ethyl acetate. The organic layer was washed with brine, dried over anhydrous magnesium sulfate, and then filtered. The resulting filtrate was concentrated under reduced pressure. The resulting residue was purified by silica gel column chromatography (n-hexane/ethyl acetate/=1/2, v/v) to give 9.7 mg of the titled compound. (Yield: 23.8%)

¹H-NMR (CDCl₃) δ 8.80 (d, 1H), 8.13-8.09 (m, 1H), 7.64 (d, 1H), 7.49-7.44 (m, 1H), 7.31 (d, 1H), 7.26-7.24 (m, 1H), 7.06 (d, 1H), 6.99-6.91 (m, 2H), 5.42-5.30 (bs, 1H), 4.13-4.08 (m, 1H), 3.21-3.19 (m, 1H), 2.04-2.00 (m, 1H), 1.88-1.64 (m, 11H), 1.36 (d, 3H)

### Example 29. 6-fluoro-N-((R)-1-((cis)-4-(6-fluoroquinolin-4-yl)cyclohexyl)propan-2-yl)benzo[d]oxazol-2-amine

The titled compound (3.9 mg) was prepared in accordance with the same procedures as in Example 28, using 2-chloro-6-fluoro-1,3-benzoxazole (16 mg) instead of 2-chloro-4-fluoro-1,3-benzoxazole. (Yield: 9.5%)

¹H-NMR (CDCl₃) δ 8.80 (d, 1H), 8.13-8.10 (m, 1H), 7.64 (d, 1H), 7.49-7.47 (m, 1H), 7.31 (d, 1H), 7.26-7.24 (m, 1H), 7.01 (d, 1H), 6.93-6.88 (m, 1H), 4.74 (m, 1H), 4.05-4.01 (m, 1H), 3.21-3.19 (m, 1H), 2.04-2.00 (m, 1H), 1.88-1.64 (m, 11H), 1.36 (d, 3H)

### Example 30. N-((R)-1-((cis)-4-(6-fluoroquinolin-4-yl)cyclohexyl)propan-2-yl)oxazolo[5,4-b]pyridin-2-amine

(R)-1-((cis)-4-(6-fluoroquinolin-4-yl)cyclohexyl)propan-2-amine hydrochloride (50 mg) prepared in Preparation 1, 2-chlorooxazolo[5,4-b]pyridine (24 mg) and potassium carbonate (43 mg) were dissolved in N,N-dimethylformamide (1.0 ml) and then triethylamine (40 μL) was slowly added thereto. The reaction mixture was stirred at 80° C. for 12 hours and then cooled to room temperature. The reaction was quenched by adding water to the reaction mixture, which was then extracted with ethyl acetate. The organic layer was washed with brine, dried over anhydrous magnesium sulfate, and then filtered. The resulting filtrate was concentrated under reduced pressure. The resulting residue was purified by silica gel column chromatography (n-hexane/ethyl acetate/=1/2, v/v) to give 40.1 mg of the titled compound. (Yield: 64.0%)

¹H-NMR (CDCl₃) δ 8.80 (d, 1H), 8.12-8.10 (m, 1H), 7.94 (d, 1H), 7.65 (d, 1H), 7.57 (d, 1H), 7.46 (t, 1H), 7.29 (d, 1H), 7.13 (t, 1H), 5.80 (d, 1H), 4.15-4.07 (m, 1H), 3.21-3.19 (m, 1H), 2.05 (s, 1H), 1.82-1.70 (m, 10H), 1.40 (d, 3H)

### Example 31. 6-chloro-N-((R)-1-((cis)-4-(6-fluoroquinolin-4-yl)cyclohexyl)propan-2-yl)oxazolo[4,5-b]pyridin-2-amine

(R)-1-((cis)-4-(6-fluoroquinolin-4-yl)cyclohexyl)propan-2-amine hydrochloride (50 mg) prepared in Preparation 1, 2,6-dichlorooxazolo[4,5-b]pyridine (30 mg), and potassium carbonate (42 mg) were dissolved in N,N-dimethylformamide (1.0 ml) and then triethylamine (21 μL) was slowly added thereto. The reaction mixture was stirred at 80° C. for 12 hours and then cooled to room temperature. The reaction was quenched by adding water to the reaction mixture, which was then extracted with ethyl acetate. The organic layer was washed with brine, dried over anhydrous magnesium sulfate, and then filtered. The resulting filtrate was concentrated under reduced pressure. The resulting residue was purified by silica gel column chromatography (n-hexane/ethyl acetate/=1/2, v/v) to give 16 mg of the titled compound. (Yield: 23.5%)

¹H-NMR (CDCl₃) δ 8.80 (d, 1H), 8.20 (s, 1H), 8.14-8.10 (m, 1H), 7.64 (d, 1H), 7.48-7.46 (m, 1H), 7.31 (d, 1H), 7.26 (s, 1H), 6.15 (m, 1H), 4.12 (m, 1H), 3.21 (m, 1H), 2.04-2.00 (m, 1H), 1.88-1.64 (m, 11H), 1.41 (d, 3H)

### Example 32. N-((R)-1-((cis)-4-(6-fluoroquinolin-4-yl)cyclohexyl)propan-2-yl)oxazolo[4,5-b]pyridin-2-amine

The titled compound (9.2 mg) was prepared in accordance with the same procedures as in Example 31, using 2-chlorooxazolo[4,5-b]pyridine (24 mg) instead of 2,6-dichlorooxazolo[4,5-b]pyridine. (Yield: 14.7%).

¹H-NMR (CDCl₃) δ 8.80 (d, 1H), 8.23 (d, 1H), 8.16-8.12 (m, 1H), 7.65 (d, 1H), 7.49-7.42 (m, 2H), 7.34 (d, 1H), 6.94-6.91 (m, 1H), 5,72 (m, 1H), 4.13 (m, 1H), 3.24-3.21 (m, 1H), 2.04-2.00 (m, 1H), 1.98-1.75 (m, 11H), 1.40 (d, 3H)

### Example 33. 6-fluoro-N-((R)-1-((cis)-4-(6-fluoroquinolin-4-yl)cyclohexyl)propan-2-yl)isoquinolin-1-amine

(R)-1-((cis)-4-(6-fluoroquinolin-4-yl)cyclohexyl)propan-2-amine hydrochloride (20 mg) prepared in Preparation 1 and 1-chloro-6-fluoro-isoquinoline (13 mg) were dissolved in 1,4-dioxane (1.0 ml) and then N,N-diisopropylethylamine (35 μL) was slowly added thereto. The reaction mixture was stirred at 80° C. for 12 hours and then cooled to room temperature. The reaction was quenched by adding water to the reaction mixture, which was then extracted with ethyl acetate. The organic layer was washed with brine, dried over anhydrous magnesium sulfate, and then filtered. The resulting filtrate was concentrated under reduced pressure. The resulting residue was purified by silica gel column chromatography (n-hexane/ethyl acetate/=1/2, v/v) to give 1.5 mg of the titled compound. (Yield: 5.6%)

¹H-NMR (CDCl₃) δ 8.82 (d, 1H), 8.20 (dd, 1H), 8.03 (dd, 1H), 7.87(d, 1H), 7.66 (dd, 1H), 7.52 (td, 1H), 7.41 (d, 1H), 7.32 (m, 2H), 6.92 (d, 1H), 4.51 (m, 1H), 3.23 (m, 1H), 2.07 (br, 1H), 1.95-1.76 (m, 8H), 1.38(d, 3H), 1.26(m, 2H)

### Example 34. 6-chloro-N-((R)-1-((cis)-4-(6-fluoroquinolin-4-yl)cyclohexyl)propan-2-yl)isoquinolin-1-amine

The titled compound (1.4 mg) was prepared in accordance with the same procedures as in Example 33, using 1,6-dichloroisoquinoline (14 mg) instead of 1-chloro-6-fluoro-isoquinoline. (Yield: 5.1%)

¹H-NMR (CDCl₃) δ 8.84 (d, 1H), 8.19 (dd, 1H), 7.98 (dd, 1H), 7.74 (d, 1H), 7.68 (m, 2H), 7.52 (td, 1H), 7.48 (dd, 1H), 7.41 (d, 1H), 6.87 (d, 1H), 4.51 (m, 1H), 3.22 (m, 1H), 2.07 (br, 1H), 1.95-1.76 (m, 8H), 1.38 (d, 3H), 1.26 (m, 2H)

### Example 35. 4-chloro-N-((R)-1-((cis)-4-(6-fluoroquinolin-4-yl)cyclohexyl)propan-2-yl)benzo[d]thiazol-2-amine

(R)-1-((cis)-4-(6-fluoroquinolin-4-yl)cyclohexyl)propan-2-amine hydrochloride (50 mg) prepared in Preparation 1 and 2,4-dichlorobenzothiazole (32 mg) were dissolved in tetrahydrofuran (1.0 ml). The resulting solution was placed in a sealed tube and then triethylamine (110 μL) was slowly added thereto. After sealing the sealed tube, the reaction mixture was stirred at 120° C. for 12 hours and then cooled to room temperature. The reaction was quenched by adding water to the reaction mixture, which was then extracted with ethyl acetate. The organic layer was washed with brine, dried over anhydrous magnesium sulfate, and then filtered. The resulting filtrate was concentrated under reduced pressure. The resulting residue was purified by silica gel column chromatography (n-hexane/ethyl acetate/=1/2, v/v) to give 15 mg of the titled compound. (Yield: 21.3%)

¹H-NMR (CDCl₃) δ 8.80 (d, 1H), 8.13 (dd, 1H), 7.66 (dd, 1H), 7.50-7.46 (m, 2H), 7.34-7.30 (m, 2H), 7.03-6.99 (m, 1H), 5.47 (br, 1H), 3.71 (br, 1H), 3.26-3.20 (m, 1H), 2.05 (br, 1H), 1.84-1.61 (m, 8H), 1.38 (d, 3H), 1.26 (d, 2H)

### Example 36. 5-fluoro-N-((R)-1-((cis)-4-(6-fluoroquinolin-4-yl)cyclohexyl)propan-2-yl)benzo[d]oxazol-2-amine

The titled compound (31 mg) was prepared as a yellow liquid in accordance with the same procedures as in Example 35, using 2-chloro-5-fluorobenzoxazole (29 mg) instead of 2,4-dichlorobenzothiazole. (Yield: 47.5%)

¹H-NMR (CDCl₃) δ 8.81 (d, 1H), 8.11 (dd, 1H), 7.66 (dd, 1H), 7.47(td, 1H), 7.33 (dd, 1H), 7.14 (dd, 1H), 7.06 (d, 1H), 6.74 (td, 1H), 4.84 (br, 1H), 4.06 (m, 1H), 3.25-3.19 (m, 1H), 2.07 (br, 1H), 1.89-1.61 (m, 8H), 1.38 (d, 3H), 1.26 (d, 2H)

### Example 37. 7-fluoro-N-((R)-1-((cis)-4-(6-fluoroquinolin-4-yl)cyclohexyl)propan-2-yl)benzo[d]oxazol-2-amine

The titled compound (26 mg) was prepared as a yellow liquid in accordance with the same procedures as in Example 35, using 2-chloro-7-fluorobenzoxazole (29 mg) instead of 2,4-dichlorobenzothiazole. (Yield: 39.8%)

¹H-NMR (CDCl₃) δ 8.81 (d, 1H), 8.12 (dd, 1H), 7.66 (dd, 1H), 7.47 (td, 1H), 7.33 (dd, 1H), 7.16-7.07 (m, 2H), 6.83 (m, 1H), 5.02 (br, 1H), 4.06 (m, 1H), 3.23-3.20 (m, 1H), 2.07 (br, 1H), 1.89-1.61 (m, 8H), 1.38 (d, 3H), 1.26 (d, 2H)

### Example 38. 2-chloro-N-((R)-1-((cis)-4-(6-fluoroquinolin-4-yl)cyclohexyl)propan-2-yl)quinazolin-4-amine

The titled compound (43 mg) was prepared in accordance with the same procedures as in Example 35, using 2,4-dichloroquinazoline (34 mg) instead of 2,4-dichlorobenzothiazole. (Yield: 62.4%)

¹H-NMR (CDCl₃) δ 8.81 (d, 1H), 8.12 (dd, 1H), 7.80-7.65 (m, 4H), 7.47 (td, 1H), 7.38 (dd, 1H), 5.73 (m, 1H), 4.60 (m, 1H), 3.25-3.19 (m, 1H), 1.98-1.61 (m, 9H), 1.38 (d, 3H), 1.26 (d, 2H)

### Example 39. 6-chloro-N-((R)-1-((cis)-4-(6-fluoroquinolin-4-yl)cyclohexyl)propan-2-yl)phthalazin-1-amine

(R)-1-((cis)-4-(6-fluoroquinolin-4-yl)cyclohexyl)propan-2-amine hydrochloride (50 mg) prepared in Preparation 1, 1,6-dichlorophthalazine (34 mg), and copper(1) iodide (9 mg) were dissolved in dimethyl sulfoxide (1.0 ml). The resulting solution was placed in a sealed tube and then potassium carbonate (43 mg) was added thereto. After sealing the sealed tube, the reaction mixture was stirred at 140° C. for 3 hours and then cooled to room temperature. The reaction was quenched by adding water to the reaction mixture, which was then extracted with ethyl acetate. The organic layer was washed with brine, dried over anhydrous magnesium sulfate, and then filtered. The resulting filtrate was concentrated under reduced pressure. The resulting residue was purified by silica gel column chromatography (n-hexane/ethyl acetate/=1/2, v/v) to give 5.1 mg of the titled compound as a yellow liquid. (Yield: 7.3%)

¹H-NMR (CDCl₃) δ 8.85 (br, 1H), 8.81 (d, 1H), 8.12 (br, 1H), 7.89 (d, 1H), 7.73 (br, 2H), 7.66 (d, 1H), 7.47 (td, 1H), 7.36 (br, 1H), 4.87 (br, 1H), 4.73 (br, 1H), 3.23-3.19 (br, 1H), 2.07 (br, 1H), 1.98-1.61 (m, 8H), 1.38 (d, 3H), 1.26 (d, 2H)

### Example 40. 2,7-dichloro-N-((R)-1-((cis)-4-(6-fluoroquinolin-4-yl)cyclohexyl)propan-2-yl)quinazolin-4-amine

The titled compound (37 mg) was prepared in accordance with the same procedures as in Example 35, using 2,4,7-trichloroquinazoline (40 mg) instead of 2,4-dichlorobenzothiazole. (Yield: 49.3%)

¹H-NMR (CDCl₃) δ 8.81 (d, 1H), 8.12 (dd, 1H), 7.77 (d, 1H), 7.68-7.62 (m, 2H), 7.47 (td, 1H), 7.42-7.36 (m, 2H), 5.71 ((br, 1H), 4.58 (m, 1H), 3.23-3.19 (m, 1H), 1.98-1.61 (m, 9H), 1.38 (d, 3H), 1.26 (d, 2H)

### Example 41. 2,7-dichloro-N-((R)-1-((trans)-4-(6-fluoroquinolin-4-yl)cyclohexyl)propan-2-yl)quinazolin-4-amine

(R)-1-((trans)-4-(6-fluoroquinolin-4-yl)cyclohexyl)propan-2-amine hydrochloride (50 mg) prepared in Preparation 4 and 2,4,7-trichloroquinazoline (40 mg) were dissolved in tetrahydrofuran (1.0 ml). The resulting solution was placed in a sealed tube and then triethylamine (110 μL) was added thereto. After sealing the sealed tube, the reaction mixture was stirred at 80° C. for 12 hours and then cooled to room temperature. The reaction was quenched by adding water to the reaction mixture, which was then extracted with ethyl acetate. The organic layer was washed with brine, dried over anhydrous magnesium sulfate, and then filtered. The resulting filtrate was concentrated under reduced pressure. The resulting residue was purified by silica gel column chromatography (n-hexane/ethyl acetate/=1/2, v/v) to give 40 mg of the titled compound as a white solid. (Yield: 54.1%)

¹H-NMR (CDCl₃) δ 8.79 (d, 1H), 8.12 (dd, 1H), 7.77 (d, 1H), 7.68-7.62 (m, 2H), 7.47 (td, 1H), 7.42-7.36 (m, 2H), 5.58 (br, 1H), 4.72 (m, 1H), 3.23-3.19 (m, 1H), 2.20 (br, 1H), 2.06-1.98 (m, 2H), 1.68-1.52 (m, 6H), 1.38 (d, 3H), 1.26 (d, 2H)

### Example 42. 7-fluoro-N-((R)-1-((cis)-4-(6-fluoroquinolin-4-yl)cyclohexyl)propan-2-yl)-6-methoxybenzo[d]oxazol-2-amine

The titled compound (39 mg) was prepared as a white solid in accordance with the same procedures as in Example 35, using 2-chloro-7-fluoro-6-methoxy-1,3-benzoxazole (34 mg) instead of 2,4-dichlorobenzothiazole. (Yield: 56.0%)

¹H-NMR (CDCl₃) δ 8.78 (d, 1H), 8.11 (dd, 1H), 7.66 (dd, 1H), 7.47 (td, 1H), 7.30 (d, 1H), 7.02 (d, 1H), 6.80 (t, 1H), 5.26 (br, 1H), 4.02 (m, 1H), 3.89 (s, 3H), 3.20 (m, 1H), 1.81-1.74 (m, 8H), 1.36 (d, 3H), 1.26 (d, 2H)

### Example 43. 6-chloro-5-fluoro-N-((R)-1-((cis)-4-(6-fluoroquinolin-4-yl)cyclohexyl)propan-2-yl)benzo[d]oxazol-2-amine

The titled compound (35 mg) was prepared as a white solid in accordance with the same procedures as in Example 35, using 2,6-dichloro-5-fluoro-1,3-benzoxazole (35 mg) instead of 2,4-dichlorobenzothiazole. (Yield: 51.0%)

¹H-NMR (CDCl₃) δ 8.79 (d, 1H), 8.12 (dd, 1H), 7.65 (dd, 1H), 7.48 (td, 1H), 7.30-7.26 (m, 2H), 7.11 (d, 1H), 5.43 (br, 1H), 4.03 (m, 1H), 3.21 (m, 1H), 1.81-1.74 (m, 9H), 1.36 (d, 3H), 1.26 (d, 2H)

### Example 44. 6-ethoxy-N-((R)-1-((cis)-4-(6-fluoroquinolin-4-yl)cyclohexyl)propan-2-yl)benzo[d]oxazol-2-amine

The titled compound (4.6 mg) was prepared in accordance with the same procedures as in Example 35, using 2-chloro-6-ethoxy-1,3-benzoxazole (34 mg) instead of 2,4-dichlorobenzothiazole. (Yield: 6.6%)

¹H-NMR (CDCl₃) δ 8.81 (d, 1H), 8.12 (dd, 1H), 7.65 (dd, 1H), 7.47 (td, 1H), 7.32 (d, 1H), 7.27-7.22 (m, 1H), 6.87 (s, 1H), 6.77 (dd, 1H), 4.05-4.00 (m, 3H), 3.22 (m, 1H), 2.08 (br, 1H), 1.83-1.65 (m, 8H), 1.42 (t, 3H), 1.38 (d, 3H)

### Example 45. 6-(2-fluoroethoxy)-N-((R)-1-((cis)-4-(6-fluoroquinolin-4-yl)cyclohexyl)propan-2-yl)benzo[d]oxazol-2-amine

The titled compound (36.6 mg) was prepared in accordance with the same procedures as in Example 35, using 2-chloro-6-(2-fluoroethoxy)-1,3-benzoxazole (37 mg) instead of 2,4-dichlorobenzothiazole. (Yield: 50.7%)

¹H-NMR (CDCl₃) δ 8.79 (d, 1H), 8.12 (dd, 1H), 7.64 (dd, 1H), 7.47 (td, 1H), 7.32 (d, 1H), 7.27-7.23 (m, 1H), 6.91 (s, 1H), 6.77 (d, 1H), 5.06 (br, 1H), 4.81 (d, 1H), 4.69 (d, 1H), 4.24 (d, 1H), 4.17 (d, 1H), 4.05-4.00 (m, 1H), 3.21 (m, 1H), 2.08 (br, 1H), 1.83-1.65 (m, 10H), 1.37 (d, 3H)

### Example 46. 6,7-dichloro-N-((R)-1-((cis)-4-(6-fluoroquinolin-4-yl)cyclohexyl)propan-2-yl)benzo[d]oxazol-2-amine

The titled compound (5.1 mg) was prepared in accordance with the same procedures as in Example 35, using 2,6,7-trichloro-1,3-benzoxazole (38 mg) instead of 2,4-dichlorobenzothiazole. (Yield: 7.0%)

¹H-NMR (CDCl₃) δ 8.80 (d, 1H), 8.13 (dd, 1H), 7.64 (dd, 1H), 7.48 (td, 1H), 7.32 (d, 1H), 7.27-7.23 (m, 1H), 7.17 (d, 1H), 4.95 (br, 1H), 4.06 (m, 1H), 3.24 (m, 1H), 2.08 (br, 1H), 1.83-1.65 (m, 10H), 1.37 (d, 3H)

### Example 47. 6, 7-difluoro-N-((R)-1-((cis)-4-(6-fluoroquinolin-4-yl)cyclohexyl)propan-2-yl)benzo[d]oxazol-2-amine

The titled compound (51.5 mg) was prepared in accordance with the same procedures as in Example 35, using 2-chloro-6,7-difluoro-1,3-benzoxazole (33 mg) instead of 2,4-dichlorobenzothiazole. (Yield: 75.6%)

¹H-NMR (CDCl₃) δ 8.79 (d, 1H), 8.12 (dd, 1H), 7.64 (dd, 1H), 7.48 (td, 1H), 7.32 (d, 1H), 7.02-6.94 (m, 2H), 5.38 (d, 1H), 4.04 (m, 1H), 3.21 (m, 1H), 2.08 (br, 1H), 1.81-1.71 (m, 10H), 1.38 (d, 3H)

### Example 48. 2-chloro-6-fluoro-N-((R)-1-((trans)-4-(6-fluoroquinolin-4-yl)cyclohexyl)propan-2-yl)quinazolin-4-amine

(R)-1-((trans)-4-(6-fluoroquinolin-4-yl)cyclohexyl)propan-2-amine hydrochloride (30 mg) prepared in Preparation 4 and 2,4-dichloro-6-fluoroquinazoline (30 mg) were dissolved in 1,4-dioxane (2.0 ml). The resulting solution was placed in a sealed tube and then triethylamine (45 μL) was added thereto. After sealing the sealed tube, the reaction mixture was stirred at 100° C. for 4 hours and then cooled to room temperature. The reaction was quenched by adding water to the reaction mixture, which was then extracted with ethyl acetate. The organic layer was washed with brine, dried over anhydrous magnesium sulfate, and then filtered. The resulting filtrate was concentrated under reduced pressure. The resulting residue was purified by silica gel column chromatography (n-hexane/ethyl acetate/=1/2, v/v) to give 12 mg of the titled compound. (Yield: 27.7%)

¹H-NMR (CDCl₃) δ 8.78 (d, 1H), 8.12 (dd, 1H), 7.78 (dd, 1H), 7.66 (dd, 1H), 7.51-7.46 (m, 2H), 7.36 (d, 1H), 7.25 (m, 1H), 5.62 (d, 1H), 4.70 (m, 1H), 3.18 (m, 1H), 2.22 (br, 1H), 2.06-1.97 (m, 4H), 1.68 (br, 2H), 1.58-1.52 (m, 4H), 1.38 (d, 3H)

### Example 49. 2,6-dichloro-N-((R)-1-((trans)-4-(6-fluoroquinolin-4-yl)cyclohexyl)propan-2-yl)quinazolin-4-amine

The titled compound (12 mg) was prepared in accordance with the same procedures as in Example 48, using 2,4,6-trichloroquinazoline (33 mg) instead of 2,4-dichloro-6-fluoroquinazoline. (Yield: 25.8%)

¹H-NMR (CDCl₃) δ 8.78 (d, 1H), 8.12 (dd, 1H), 7.73-7.65 (m, 4H), 7.48 (td, 1H), 7.25 (m, 1H), 5.69 (d, 1H), 4.70(m, 1H), 3.18 (m, 1H), 2.19 (br, 1H), 2.06-1.97 (m, 4H), 1.68 (br, 2H), 1.58 -1.52 (m, 4H), 1.38 (d, 3H)

### Example 50. 2-chloro-8-fluoro-N-((R)-1-((trans)-4-(6-fluoroquinolin-4-yl)cyclohexyl)propan-2-yl)quinazolin-4-amine

The titled compound (10.3 mg) was prepared in accordance with the same procedures as in Example 48, using 2,4-dichloro-8-fluoroquinazoline (30 mg) instead of 2,4-dichloro-6-fluoroquinazoline. (Yield: 23.7%)

¹H-NMR (CDCl₃) δ 8.79 (d, 1H), 8.13 (dd, 1H), 7.67 (dd, 1H), 7.51-7.39 (m, 4H), 7.25 (m, 1H), 5.75 (d, 1H), 4.75 (m, 1H), 3.15 (m, 1H), 2.19 (d, 1H), 2.06-1.97 (m, 4H), 1.68 (m, 2H), 1.58-1.52 (m, 4H), 1.38 (d, 3H)

### Example 51. 2,8-dichloro-N-((R)-1-((trans)-4-(6-fluoroquinolin-4-yl)cyclohexyl)propan-2-yl)quinazolin-4-amine

The titled compound (12.1 mg) was prepared in accordance with the same procedures as in Example 48, using 2,4,8-trichloroquinazoline (33 mg) instead of 2,4-dichloro-6-fluoroquinazoline. (Yield: 26.9%)

¹H-NMR (CDCl₃) δ 8.79 (d, 1H), 8.13 (dd, 1H), 7.84 (d, 1H), 7.67-7.63 (m, 2H), 7.45 (m, 1H), 7.37 (td, 1H), 7.25 (m, 1H), 5.77 (d, 1H), 4.75 (m, 1H), 3.15 (m, 1H), 2.19 (d, 1H), 2.06-1.97 (m, 4H), 1.68 (m, 2H), 1.58-1.52 (m, 4H), 1.38 (d, 3H)

### Example 52. 2-chloro-N-((R)-1-((trans)-4-(6-fluoroquinolin-4-yl)cyclohexyl)propan-2-yl)-8-methoxyquinazolin-4-amine

The titled compound (9.6 mg) was prepared in accordance with the same procedures as in Example 48, using 2,4-dichloro-8-methoxyquinazoline (32 mg) instead of 2,4-dichloro-6-fluoroquinazoline. (Yield: 21.6%)

¹H-NMR (CDCl₃) δ 8.79 (d, 1H), 8.11 (dd, 1H), 7.67 (dd, 1H), 7.47 (td, 1H), 7.39 (td, 1H), 7.25 (m, 2H), 7.13 (d, 1H), 5.63 (d, 1H), 4.72 (m, 1H), 4.02 (s, 3H), 3.15 (m, 1H), 2.19 (d, 1H), 2.06-1.97 (m, 4H), 1.68 (m, 2H), 1.58-1.52 (m, 4H), 1.38 (d, 3H)

### Example 53. 2-chloro-N-((R)-1-((trans)-4-(6-fluoroquinolin-4-yl)cyclohexyl)propan-2-yl)-8-(trifluoromethoxy)quinazolin-4-amine

The titled compound (10.8 mg) was prepared in accordance with the same procedures as in Example 48, using 2,4-dichloro-8-(trifluoromethoxy)quinazoline (40 mg) instead of 2,4-dichloro-6-fluoroquinazoline. (Yield: 21.8%)

¹H-NMR (CDCl₃) δ 8.79 (d, 1H), 8.11 (dd, 1H), 7.69-7.65 (m, 3H), 7.49-7.43 (m, 2H), 7.25 (m, 1H), 5.74 (d, 1H), 4.70 (m, 1H), 3.18 (m, 1H), 2.19 (d, 1H), 2.06-1.97 (m, 4H), 1.68 (m, 2H), 1.58-1.52 (m, 4H), 1.38 (d, 3H)

### Example 54. 2-chloro-7-fluoro-N-((R)-1-((trans)-4-(6-fluoroquinolin-4-yl)cyclohexyl)propan-2-yl)quinazolin-4-amine

The titled compound (6.9 mg) was prepared in accordance with the same procedures as in Example 48, using 2,4-dichloro-7-fluoroquinazoline (30 mg) instead of 2,4-dichloro-6-fluoroquinazoline. (Yield: 15.9%)

¹H-NMR (CDCl₃) δ 8.79 (d, 1H), 8.12 (dd, 1H), 7.73-7.64 (m, 2H), 7.47-7.40 (m, 2H), 7.25 (m, 1H), 7.23 (t, 1H), 5.61 (d, 1H), 4.72 (m, 1H), 3.18 (td, 1H), 2.19 (d, 1H), 2.06-1.97 (m, 4H), 1.68 (m, 2H), 1.58-1.52 (m, 4H), 1.38 (d, 3H)

### Example 55. 2-chloro-4-(((R)-1-((trans)-4-(6-fluoroquinolin-4-yl)cyclohexyl)propan-2-yl)amino)quinazolin-7-carbonitrile

The titled compound (4.6 mg) was prepared in accordance with the same procedures as in Example 48, using 2,4-dichloroquinazolin-7-carbonitrile (32 mg) instead of 2,4-dichloro-6-fluoroquinazoline. (Yield: 10.4%)

¹H-NMR (CDCl₃) δ 8.79 (d, 1H), 8.12 (d, 1H), 8.09 (s, 1H), 7.81 (d, 1H), 7.67-7.62 (m, 2H), 7.46 (m, 1H), 7.25 (m, 1H), 5.81 (d, 1H), 4.72 (m, 1H), 3.17 (td, 1H), 2.19 (d, 1H), 2.06-1.97 (m, 4H), 1.68 (m, 1H), 1.58-1.52 (m, 4H), 1.38 (d, 3H), 1.35 (m, 1H)

### Example 56. 7-bromo-2-chloro-N-((R)-1-((trans)-4-(6-fluoroquinolin-4-yl)cyclohexyl)propan-2-yl)quinazolin-4-amine

The titled compound (8.0 mg) was prepared in accordance with the same procedures as in Example 48, using 7-bromo-2,4-dichloroquinazoline (39 mg) instead of 2,4-dichloro-6-fluoroquinazoline. (Yield: 16.3%)

¹H-NMR (CDCl₃) δ 8.79 (d, 1H), 8.12 (t, 1H), 7.95 (s, 1H), 7.66 (d, 1H), 7.55 (m, 2H), 7.46 (t, 1H), 7.25 (m, 1H), 5.68 (d, 1H), 4.72 (m, 1H), 3.16 (td, 1H), 2.19 (d, 1H), 2.06-1.97 (m, 4H), 1.68 (m, 2H), 1.58-1.52 (m, 4H), 1.38 (d, 3H)

### Example 57. 2-chloro-N-((R)-1-((trans)-4-(6-fluoroquinolin-4-yl)cyclohexyl)propan-2-yl)-7-methylquinazolin-4-amine

The titled compound (7.1 mg) was prepared in accordance with the same procedures as in Example 48, using 2,4-dichloro-7-methylquinazoline (30 mg) instead of 2,4-dichloro-6-fluoroquinazoline. (Yield: 16.5%)

¹H-NMR (CDCl₃) δ 8.79 (d, 1H), 8.12 (t, 1H), 7.67 (d, 1H), 7.58 (m, 2H), 7.46 (t, 1H), 7.30-7.24 (m, 2H), 5.56 (d, 1H), 4.73 (m, 1H), 3.16 (td, 1H), 2.51 (s, 3H), 2.22 (d, 1H), 2.06-1.97 (m, 4H), 1.68 (m, 2H), 1.58-1.52 (m, 4H), 1.36 (d, 3H)

### Example 58. 2-chloro-N-((R)-1-((trans)-4-(6-fluoroquinolin-4-yl)cyclohexyl)propan-2-yl)-7-(trifluoromethyl)quinazolin-4-amine

The titled compound (7.7 mg) was prepared in accordance with the same procedures as in Example 48, using 2,4-dichloro-7-(trifluoromethyl)quinazoline (38 mg) instead of 2,4-dichloro-6-fluoroquinazoline. (Yield: 16.0%)

¹H-NMR (CDCl₃) δ 8.79 (d, 1H), 8.12 (t, 1H), 8.07 (s, 1H), 7.84 (d, 1H), 7.65 (t, 2H), 7.46 (t, 1H), 7.27-7.24 (m, 1H), 5.82 (d, 1H), 4.73 (m, 1H), 3.16 (td, 1H), 2.22 (d, 1H), 2.06-1.97 (m, 4H), 1.68 (m, 2H), 1.58-1.52 (m, 4H), 1.36 (d, 3H)

### Example 59. 2-chloro-N-((R)-1-((trans)-4-(6-fluoroquinolin-4-yl)cyclohexyl)propan-2-yl)-7-methoxyquinazolin-4-amine

The titled compound (9.7 mg) was prepared in accordance with the same procedures as in Example 48, using 2,4-dichloro-7-methoxyquinazoline (32 mg) instead of 2,4-dichloro-6-fluoroquinazoline. (Yield: 21.8%)

¹H-NMR (CDCl₃) δ 8.78 (d, 1H), 8.11 (t, 1H), 7.68 (d, 1H), 7.65 (d, 1H), 7.46 (td, 1H), 7.27-7.24 (m, 1H), 7.13 (s, 1H), 7.05 (d, 1H), 5.48 (d, 1H), 4.70 (m, 1H), 3.92 (s, 3H), 3.16 (td, 1H), 2.22 (d, 1H), 2.06-1.97 (m, 4H), 1.68 (m, 2H), 1.58-1.52 (m, 4H), 1.36 (d, 3H)

### Example 60. 2-chloro-6-fluoro-N-((R)-1-((cis)-4-(6-fluoroquinolin-4-yl)cyclohexyl)propan-2-yl)quinazolin-4-amine

(R)-1-((cis)-4-(6-fluoroquinolin-4-yl)cyclohexyl)propan-2-amine hydrochloride (50 mg) prepared in Preparation 1 and 2,4-dichloro-6-fluoroquinazoline (34 mg) were dissolved in tetrahydrofuran (1.0 ml). The resulting solution was placed in a sealed tube and then triethylamine (110 μL) was added thereto. After sealing the sealed tube, the reaction mixture was stirred at 80° C. for 12 hours and then cooled to room temperature. The reaction was quenched by adding water to the reaction mixture, which was then extracted with ethyl acetate. The organic layer was washed with brine, dried over anhydrous magnesium sulfate, and then filtered. The resulting filtrate was concentrated under reduced pressure. The resulting residue was purified by silica gel column chromatography (n-hexane/ethyl acetate/=1/2, v/v) to give 38 mg of the titled compound as a white solid. (Yield: 52.4%)

¹H-NMR (CDCl₃) δ 8.81 (d, 1H), 8.12 (t, 1H), 7.80 (d, 1H), 7.67 (d, 1H), 7.52-7.46 (m, 2H), 7.38-7.35 (m, 2H), 5.68 (d, 1H), 4.60 (m, 1H), 3.23 (td, 1H), 1.99-1.62 (m, 11H), 1.39 (d, 3H)

### Example 61. 2,6-dichloro-N-((R)-1-((cis)-4-(6-fluoroquinolin-4-yl)cyclohexyl)propan-2-yl)quinazolin-4-amine

The titled compound (45.2 mg) was prepared as a white solid in accordance with the same procedures as in Example 60, using 2,4,6-trichloroquinazoline (37 mg) instead of 2,4-dichloro-6-fluoroquinazoline. (Yield: 60.3%)

¹H-NMR (CDCl₃) δ 8.81 (d, 1H), 8.12 (t, 1H), 7.73-7.65 (m, 4H), 7.46 (t, 1H), 7.37 (s, 1H), 5.79 (d, 1H), 4.59 (m, 1H), 3.22 (td, 1H), 1.99-1.62 (m, 11H), 1.39 (d, 3H)

### Example 62. 2-chloro-7-fluoro-N-((R)-1-((cis)-4-(6-fluoroquinolin-4-yl)cyclohexyl)propan-2-yl)quinazolin-4-amine

The titled compound (40.7 mg) was prepared as a pale yellow liquid in accordance with the same procedures as in Example 60, using 2,4-dichloro-7-fluoroquinazoline (37 mg) instead of 2,4-dichloro-6-fluoroquinazoline. (Yield: 56.3%)

¹H-NMR (CDCl₃) δ 8.81 (d, 1H), 8.12 (t, 1H), 7.73 (dd, 1H), 7.65 (dd, 1H), 7.46 (td, 1H), 7.41 (dd, 1H), 7.37 (d, 1H), 7.22 (td, 1H), 5.71 (d, 1H), 4.59 (m, 1H), 3.22 (td, 1H), 1.99-1.62 (m, 11H), 1.39 (d, 3H)

### Example 63. 2-chloro-4-(((R)-1-((cis)-4-(6-fluoroquinolin-4-yl)cyclohexyl)propan-2-yl)amino)quinazolin-7-carbonitrile

The titled compound (33 mg) was prepared as a pale yellow liquid in accordance with the same procedures as in Example 60, using 2,4-dichloroquinazolin-7-carbonitrile (39 mg) instead of 2,4-dichloro-6-fluoroquinazoline. (Yield: 45.0%)

¹H-NMR (CDCl₃) δ 8.81 (d, 1H), 8.12 (t, 1H), 8.09 (s, 1H), 7.81 (d, 1H), 7.68-7.62 (m, 2H), 7.46 (td, 1H), 7.36 (d, 1H), 5.89 (d, 1H), 4.61 (m, 1H), 3.23 (m, 1H), 1.99-1.62 (m, 11H), 1.39 (d, 3H)

### Example 64. 7-bromo-2-chloro-N-((R)-1-((cis)-4-(6-fluoroquinolin-4-yl)cyclohexyl)propan-2-yl)quinazolin-4-amine

The titled compound (53.4 mg) was prepared as a pale yellow liquid in accordance with the same procedures as in Example 60, using 7-bromo-2,4-dichloroquinazoline (48 mg) instead of 2,4-dichloro-6-fluoroquinazoline. (Yield: 65.3%)

¹H-NMR (CDCl₃) δ 8.81 (d, 1H), 8.12 (t, 1H), 7.94 (s, 1H), 7.67 (d, 1H), 7.59-7.55 (m, 2H), 7.46 (td, 1H), 7.36 (d, 1H), 5.89 (d, 1H), 4.58 (m, 1H), 3.22 (m, 1H), 1.99-1.62 (m, 11H), 1.39 (d, 3H)

### Example 65. 2-chloro-N-((R)-1-((cis)-4-(6-fluoroquinolin-4-yl)cyclohexyl)propan-2-yl)-7-methylquinazolin-4-amine

The titled compound (49.5 mg) was prepared as a pale yellow liquid in accordance with the same procedures as in Example 60, using 2,4-dichloro-7-methylquinazoline (37 mg) instead of 2,4-dichloro-6-fluoroquinazoline. (Yield: 69.3%)

¹H-NMR (CDCl₃) δ 8.81 (d, 1H), 8.12 (t, 1H), 7.67 (dd, 1H), 7.59-7.56 (m, 2H), 7.47 (td, 1H), 7.36 (d, 1H), 7.28 (m, 1H), 5.67 (d, 1H), 4.58 (m, 1H), 3.21 (m, 1H), 2.51 (s, 3H), 1.99-1.62 (m, 11H), 1.39 (d, 3H)

### Example 66. 2-chloro-N-((R)-1-((cis)-4-(6-fluoroquinolin-4-yl)cyclohexyl)propan-2-yl)-7-(trifluoromethyl)quinazolin-4-amine

The titled compound (44.8 mg) was prepared as a pale yellow liquid in accordance with the same procedures as in Example 60, using 2,4-dichloro-7-(trifluoromethyl)quinazoline (46 mg) instead of 2,4-dichloro-6-fluoroquinazoline. (Yield: 56.0%)

¹H-NMR (CDCl₃) δ 8.81 (d, 1H), 8.12 (t, 1H), 8.10 (s, 1H), 7.86 (d, 1H), 7.68-7.63 (m, 2H), 7.47 (td, 1H), 7.36 (d, 1H), 5.91 (d, 1H), 4.62 (m, 1H), 3.23 (m, 1H), 1.99-1.62 (m, 11H), 1.39 (d, 3H)

### Example 67. 2-chloro-N-((R)-1-((cis)-4-(6-fluoroquinolin-4-yl)cyclohexyl)propan-2-yl)-7-methoxyquinazolin-4-amine

The titled compound (52.1 mg) was prepared as a pale yellow liquid in accordance with the same procedures as in Example 60, using 2,4-dichloro-7-methoxyquinazoline (39 mg) instead of 2,4-dichloro-6-fluoroquinazoline. (Yield: 70.2%)

¹H-NMR (CDCl₃) δ 8.81 (d, 1H), 8.12 (t, 1H), 7.67 (dd, 1H), 7.58 (d, 1H), 7.46 (td, 1H), 7.37 (d, 1H), 7.13 (d, 1H), 7.04 (dd, 1H), 5.58 (d, 1H), 4.60 (m, 1H), 3.92 (s, 3H), 3.22 (m, 1H), 1.99-1.62 (m, 11H), 1.39 (d, 3H)

### Example 68. 2,8-dichloro-N-((R)-1-((cis)-4-(6-fluoroquinolin-4-yl)cyclohexyl)propan-2-yl)quinazolin-4-amine

The titled compound (49.5 mg) was prepared as a pale yellow liquid in accordance with the same procedures as in Example 60, using 2,4,8-trichloroquinazoline (40 mg) instead of 2,4-dichloro-6-fluoroquinazoline. (Yield: 66.1%)

¹H-NMR (CDCl₃) δ 8.81 (d, 1H), 8.12 (t, 1H), 7.83 (d, 1H), 7.68-7.73 (m, 2H), 7.46 (td, 1H), 7.39-7.35 (m, 2H), 5.82 (d, 1H), 4.60 (m, 1H), 3.22 (m, 1H), 1.99-1.62 (m, 11H), 1.39 (d, 3H)

### Example 69. 2-chloro-8-fluoro-N-((R)-1-((cis)-4-(6-fluoroquinolin-4-yl)cyclohexyl)propan-2-yl)quinazolin-4-amine

The titled compound (44.7 mg) was prepared as a pale yellow liquid in accordance with the same procedures as in Example 60, using 2,4-dichloro-8-fluoroquinazoline (37 mg) instead of 2,4-dichloro-6-fluoroquinazoline. (Yield: 61.8%)

¹H-NMR (CDCl₃) δ 8.81 (d, 1H), 8.12 (t, 1H), 7.67 (dd, 1H), 7.49-7.44 (m, 3H), 7.40-7.37 (m, 2H), 5.82 (d, 1H), 4.60 (m, 1H), 3.22 (m, 1H), 1.99-1.62 (m, 11H), 1.39 (d, 3H)

### Example 70. 2-chloro-N-((R)-1-((cis)-4-(6-fluoroquinolin-4-yl)cyclohexyl)propan-2-yl)-8-methoxyquinazolin-4-amine

The titled compound (36.1 mg) was prepared as a pale yellow liquid in accordance with the same procedures as in Example 60, using 2,4-dichloro-8-methoxyquinazoline (39 mg) instead of 2,4-dichloro-6-fluoroquinazoline. (Yield: 48.6%)

¹H-NMR (CDCl₃) δ 8.81 (d, 1H), 8.12 (t, 1H), 7.67 (dd, 1H), 7.48 (td, 1H), 7.44-7.37 (m, 2H), 7.22 (d, 1H), 7.13 (d, 1H), 5.65 (d, 1H), 4.58 (m, 1H), 4.01 (s, 3H), 3.22 (m, 1H), 1.99-1.62 (m, 11H), 1.39 (d, 3H)

### Example 71. 2-chloro-N-((R)-1-((cis)-4-(6-fluoroquinolin-4-yl)cyclohexyl)propan-2-yl)-8-(trifluoromethoxy)quinazolin-4-amine

The titled compound (58.4 mg) was prepared as a pale yellow liquid in accordance with the same procedures as in Example 60, using 2,4-dichloro-8-(trifluoromethoxy)quinazoline (49 mg) instead of 2,4-dichloro-6-fluoroquinazoline. (Yield: 70.7%)

¹H-NMR (CDCl₃) δ 8.81 (d, 1H), 8.12 (t, 1H), 7.68-7.64 (m, 3H), 7.49-7.41 (m, 2H), 7.37 (d, 1H), 5.86 (d, 1H), 4.59 (m, 1H), 3.22 (m, 1H), 1.99-1.62 (m, 11H), 1.39 (d, 3H)

### Experimental Example 1: Analyses of IDO1 Cellular Activities Using Hela Cells

Hela cells were seeded at a density of 20000 cells/well in 100 μl of a culture medium (EMEM supplemented with 10% FBS, penicillin 100 U/ml, and streptomycin 100 μg/ml) in a tissue culture-treated 96 well plate. The cells were cultured in a 5% carbon dioxide incubator for 24 hours, treated with recombinant human interferon gamma at the concentration of 50 ng/ml, and then cultured for 48 hours in a 5% carbon dioxide incubator to induce IDO expression.

The activities were measured using IDO1 cellular activity-measuring substrate (IDO1 cellular activity quickDetect supplements catalog: #62000-2, BPS Bioscience). Specifically, an assay medium was prepared by diluting IDO1 assay medium substrate 1 and IDO1 assay medium supplement 2 in a culture medium at a ratio of 1:100, respectively. The test compounds were diluted to the predetermined concentrations in the fresh assay medium. After removing the culture medium using a multi-pipet, 200 μl of each assay medium containing the test material was added into the respective well, followed by incubating in a 5% carbon dioxide incubator for 24 hours. The next day, 140 μl of culture medium from each well was transferred to a new 96-well plate. 10 μl of 6.1N trichloroacetic acid was added into each well, which was then cultured in a 50° C. incubator for 30 minutes. The plate was centrifuged at 2500 rpm for 10 minutes to settle any sediment. A detection reagent solution was prepared by diluting the detection reagent (component D kit, catalog: #62000-2, BPS Bioscience) 50-fold in acetic acid. 100 μl of the supernatant taken from the centrifuged plate was transferred to a new transparent 96-well plate and then 100 μl of the detection reagent solution was added thereto. After reacting at room temperature for 10 minutes, absorbance was measured at 480 nm wavelength. For analysis of the results, the absorbance (At) of the well containing Hela cells in which IDO protein expression was induced without treatment of an inhibitor was 100%; and the absorbance (Ab) of the well containing Hela cells in which IDO protein expression was not induced was 0. The % absorbance was calculated according to the following equation: % Absorbance=(A−Ab)/(At−Ab), A=absorbance of the well treated with the test compound.

The results obtained by calculating the 50% inhibitory concentration (C₅₀) of each compound, from the absorbance values obtained as described above, are shown in Table 1 below.

TABLE 1

| | Inhibitory Activity |
|---|---|
| | (IC₅₀, nM) |
| Example 1 | 16 |
| Example 2 | 8.8 |
| Example 3 | 14 |
| Example 4 | 11 |
| Example 5 | 16 |
| Example 6 | 17 |
| Example 7 | 19 |
| Example 8 | 5.9 |
| Example 9 | 18 |
| Example 10 | 10.2 |
| Example 11 | 18.7 |
| Example 12 | 7.2 |
| Example 13 | 30 |
| Example 14 | 1 |
| Example 15 | 5.1 |
| Example 16 | 4 |
| Example 17 | 19 |
| Example 18 | 7.5 |
| Example 19 | 14 |
| Example 20 | 24 |
| Example 21 | 18 |
| Example 22 | 16 |
| Example 23 | 27 |
| Example 24 | 17 |
| Example 25 | 4.4 |
| Example 26 | 15 |
| Example 27 | 23 |
| Example 28 | 12 |
| Example 29 | 10 |
| Example 30 | 23.5 |
| Example 31 | 17.5 |
| Example 32 | 11.4 |

From the results of Table 1, it can be seen that the compound of the present invention exhibits excellent inhibitory activity against indoleamine 2,3-dioxygenase.

### Experimental Example 2: Analyses of IDO1 Cellular Activities Using HEK293 Cells

HEK293 cells were seeded at a density of 30000 cells/well in 100 μl of a culture medium (DMEM supplemented with 10% FBS, penicillin 100 U/ml, and streptomycin 100 μg/ml) in a tissue culture-treated 96 well plate. The cells were cultured in a 5% carbon dioxide incubator for 24 hours, transfected with the IDO1 expression vector (component A in IDO1 cell-based assay kit, catalog #72031, BPS Bioscience) using Lipofectamine 2000 (Life Technologies, #11668027), and then cultured for 24 hours in a 5% carbon dioxide incubator to express the IDO protein.

The activities were measured using a IDO1 cell-based assay kit (catalog #72031, BPS Bioscience). Specifically, an assay medium was prepared by diluting IDO1 assay medium substrate 1 and IDO1 assay medium supplement 2 in a culture medium at a ratio of 1:100, respectively. The test compounds were diluted to the predetermined concentrations in the fresh assay medium. After removing the culture medium using a multi-pipet, 200 μl of each assay medium containing the test material was added into respective well, followed by incubating in a 5% carbon dioxide incubator for 24 hours. The next day, 140 μl of culture medium from each well was transferred to a new 96-well plate. 10 μl of 6.1N trichloroacetic acid was added into each well, which was then cultured in a 50° C. incubator for 30 minutes. The plate was centrifuged at 2500 rpm for 10 minutes to settle any sediment. A detection reagent solution was prepared by diluting the detection reagent (component D in IDO1 cell-based assay kit, catalog #72031 BPS Bioscience) 50-fold in acetic acid. 100 μl of the supernatant taken from the centrifuged plate was transferred to a new transparent 96-well plate and then 100 μl of the detection reagent solution was added thereto. After reacting at room temperature for 10 minutes, absorbance was measured at 480 nm wavelength. For analysis of the results, the absorbance (At) of the well containing HEK293 cells in which IDO protein was expressed without treatment of an inhibitor was 100%; and the absorbance (Ab) of the well containing HEK293 cells in which IDO protein was not expressed was 0. The % absorbance was calculated according to the following equation: % Absorbance=(A−Ab)/(At−Ab), A=absorbance of the well treated with the test compound.

The results obtained by calculating the 50% inhibitory concentration (IC₅₀ of each compound, from the absorbance values obtained as described above, are shown in Table 2 below.

TABLE 2

| | Inhibitory Activity |
|---|---|
| | (IC₅₀, nM) |
| Example 10 | 9.5 |
| Example 11 | 18.3 |
| Example 12 | 11.2 |
| Example 13 | 32.6 |
| Example 17 | 20.6 |
| Example 18 | 4 |
| Example 19 | 8 |
| Example 20 | 14.3 |
| Example 22 | 10.9 |
| Example 23 | 7.6 |
| Example 25 | 5.2 |
| Example 26 | 6.5 |
| Example 27 | 7.4 |
| Example 28 | 8.6 |
| Example 29 | 9.9 |
| Example 30 | 19.1 |
| Example 31 | 9.1 |
| Example 32 | 8.7 |
| Example 33 | 116.5 |
| Example 34 | 68.6 |
| Example 35 | 104.7 |
| Example 36 | 5.7 |
| Example 37 | 7.6 |
| Example 38 | 23.7 |
| Example 39 | 9.7 |
| Example 40 | 21.9 |
| Example 41 | 7.6 |
| Example 42 | 6.6 |
| Example 43 | 6.3 |
| Example 44 | 4.4 |
| Example 45 | 5.5 |
| Example 46 | 17.2 |
| Example 47 | 5.5 |
| Example 48 | 45.4 |
| Example 49 | 72.0 |
| Example 50 | 62.3 |
| Example 51 | 123.8 |
| Example 52 | 260.1 |
| Example 53 | 57.4 |
| Example 54 | 43.6 |
| Example 55 | 28.2 |
| Example 56 | 19.3 |
| Example 57 | 25.2 |
| Example 58 | 16.8 |
| Example 59 | 19.4 |
| Example 60 | 61.1 |
| Example 61 | 406.4 |
| Example 62 | 56.6 |
| Example 63 | 27.5 |
| Example 64 | 38.8 |
| Example 65 | 16.1 |
| Example 66 | 30.8 |
| Example 67 | 47.5 |
| Example 68 | 50.5 |
| Example 69 | 18.8 |
| Example 70 | 114.5 |
| Example 71 | 275.8 |

From the results of Table 2, it can be seen that the compound of the present invention exhibits excellent inhibitory activity against indoleamine 2,3-dioxygenase.

### Experimental Example 3: Comparative Study of Pharmacokinetics Through Oral Administration in Normal Rats

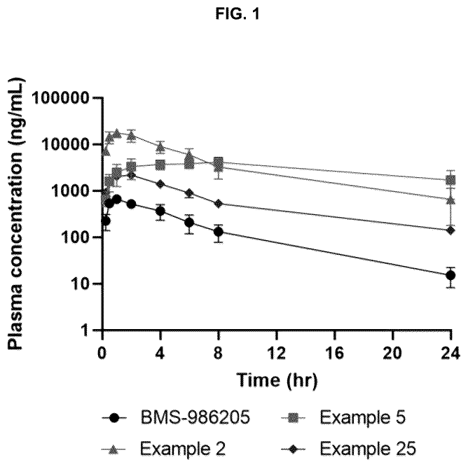

The pharmacokinetics of the compounds of Examples 2, 5 and 25 and BMS-986205 (control) were measured in rats, respectively. The compounds of Examples 2, 5 and 25 and BMS-986205 (control) were each suspended in 0.5% methyl cellulose containing 0.2% Tween 80 and then orally administered to rats at a dose of 10 mg/5 mL/kg. Blood samples were collected from the rat in predetermined times. The concentration of the compound in each sample was analyzed to obtain the blood concentration profiles (FIG. 1). The pharmacokinetic parameters obtained therefrom are shown in Table 3 below.

TABLE 3

| | | Time to | |
|---|---|---|---|
| | Maximum | maximum | |
| | plasma | plasma | Area under |
| | concentration | concentration | the curve |
| | (ng/mL) | (hr) | (ng · hr/mL) |
| BMS-986205 | 656.8 | 1 | 3976.7 |
| Example 2 | 18050.3 | 1 | 108441.1 |
| Example 5 | 4476.3 | 4 | 73460.2 |
| Example 25 | 2170.3 | 2 | 16018 |

As can be seen from the results of Table 3, the compounds of the present invention exhibit 3.3˜27.5 times higher Cmax, and 4.0˜27.3 times higher AUC, compared to the control (BMS-986205), in normal rats. These results show that the compounds of the present invention exhibit remarkably high in vivo exposure. Accordingly, the compounds of the present invention are expected to show excellent drug efficacy by exhibiting significantly higher in vivo exposure than BMS-986205 at the same dose. In addition, the compounds of the present invention are expected to show excellent safety since they can obtain similar in vivo exposure to BMS-986205 even when administered at a low dose.

1. A compound of Formula 1 or pharmaceutically acceptable salt thereof: 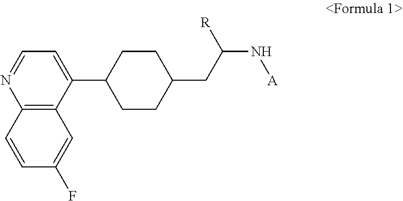
wherein,
R is a C₁˜C₆ alkyl group or a C₃˜C₁₀ cycloalkyl group,
A is a heteroaryl group selected from the group consisting of quinazolinyl, 2H-chromen-2-on-yl, benzothiazolyl, benzoxazolyl, thiazolopyridinyl, oxazolopyridinyl, isoquinolinyl, and phthalazinyl, wherein the heteroaryl group is optionally substituted with one or two substituents selected from the group consisting of halogen, C₁˜C₆ alkyl, trifluoromethyl, C₁˜C₆ alkoxy, halogeno-C₁˜C₆ alkoxy, trifluoromethoxy, and cyano.

2. The compound or pharmaceutically acceptable salt thereof as claimed in claim 1, wherein R is methyl, ethyl, or cyclopropyl.

3. The compound or pharmaceutically acceptable salt thereof as claimed in claim 1, wherein A is a quinazolinyl group substituted with one or two substituents selected from the group consisting of halogen, C₁˜C₆ alkyl, trifluoromethyl, C₁˜C₆ alkoxy, trifluoromethoxy, and cyano.

4. The compound or pharmaceutically acceptable salt thereof as claimed in claim 1, wherein A is a 2H-chromen-2-on-yl group substituted with one or two halogens.

5. The compound or pharmaceutically acceptable salt thereof as claimed in claim 1, wherein A is a benzothiazolyl group substituted with one or two substituents selected from the group consisting of halogen and C₁˜C₆ alkoxy.

6. The compound or pharmaceutically acceptable salt thereof as claimed in claim 1, wherein A is a thiazolopyridinyl group optionally substituted with halogen or C₁˜C₆ alkoxy.

7. The compound or pharmaceutically acceptable salt thereof as claimed in claim 1, wherein A is a benzoxazolyl group optionally substituted with one or two substituents selected from the group consisting of halogen, C₁˜C₆ alkoxy, and halogeno-C₁˜C₆ alkoxy.

8. The compound or pharmaceutically acceptable salt thereof as claimed in claim 1, wherein A is an oxazolopyridinyl group optionally substituted with halogen.

9. The compound or pharmaceutically acceptable salt thereof as claimed in claim 1, wherein A is an isoquinolinyl group substituted with halogen.

10. The compound or pharmaceutically acceptable salt thereof as claimed in claim 1, wherein A is a phthalazinyl group substituted with halogen.

11. The compound or pharmaceutically acceptable salt thereof as claimed in claim 1, which is selected from the group consisting of:
4-(((R)-1-((cis)-4-(6-fluoroquinolin-4-yl)cyclohexyl)propan-2-yl)amino)quinazolin-7-carbonitrile;
7-bromo-N-((R)-1-((cis)-4-(6-fluoroquinolin-4-yl)cyclohexyl)propan-2-yl)quinazolin-4-amine;
7-chloro-N-((R)-1-((cis)-4-(6-fluoroquinolin-4-yl)cyclohexyl)propan-2-yl)quinazolin-4-amine;
7-chloro-N-((R)-1-((trans)-4-(6-fluoroquinolin-4-yl)cyclohexyl)propan-2-yl)quinazolin-4-amine;
7-bromo-4-(((R)-1-((trans)-4-(6-fluoroquinolin-4-yl)cyclohexyl)propan-2-yl)amino)-2H-chromen-2-one;
7-chloro-4-(((R)-1-((trans)-4-(6-fluoroquinolin-4-yl)cyclohexyl)propan-2-yl)amino)-2H-chromen-2-one;
6-bromo-4-(((R)-1-((trans)-4-(6-fluoroquinolin-4-yl)cyclohexyl)propan-2-yl)amino)-2H-chromen-2-one;
7-chloro-4-(((R)-1-((cis)-4-(6-fluoroquinolin-4-yl)cyclohexyl)propan-2-yl)amino)-2H-chromen-2-one;
7-bromo-4-(((R)-1-((cis)-4-(6-fluoroquinolin-4-yl)cyclohexyl)propan-2-yl)amino)-2H-chromen-2-one;
7-chloro-3-fluoro-4-(((R)-1-((trans)-4-(6-fluoroquinolin-4-yl)cyclohexyl)propan-2-yl)amino)-2H-chromen-2-one;
7-bromo-3-fluoro-4-(((R)-1-((cis)-4-(6-fluoroquinolin-4-yl)cyclohexyl)propan-2-yl)amino)-2H-chromen-2-one;
7-bromo-3-fluoro-4-(((R)-1-((trans)-4-(6-fluoroquinolin-4-yl)cyclohexyl)propan-2-yl)amino)-2H-chromen-2-one;
6-chloro-N-((R)-1-((cis)-4-(6-fluoroquinolin-4-yl)cyclohexyl)propan-2-yl)benzo[d]thiazol-2-amine;
N-((R)-1-((cis)-4-(6-fluoroquinolin-4-yl)cyclohexyl)propan-2-yl)-6-methoxybenzo[d]thiazol-2-amine;
N-((R)-1-((cis)-4-(6-fluoroquinolin-4-yl)cyclohexyl)butan-2-yl)-6-methoxybenzo[d]thiazol-2-amine;
N-((S)-1-cyclopropyl-2-((cis)-4-(6-fluoroquinolin-4-yl)cyclohexyl)ethyl)-6-methoxybenzo[d]thiazol-2-amine;
4,6-difluoro-N-((R)-1-((cis)-4-(6-fluoroquinolin-4-yl)cyclohexyl)propan-2-yl)benzo[d]thiazol-2-amine;
7-chloro-N-((R)-1-((cis)-4-(6-fluoroquinolin-4-yl)cyclohexyl)propan-2-yl)benzo[d]thiazol-2-amine;
6-ethoxy-N-((R)-1-((cis)-4-(6-fluoroquinolin-4-yl)cyclohexyl)propan-2-yl)benzo[d]thiazol-2-amine;
6-ethoxy-N-((R)-1-((cis)-4-(6-fluoroquinolin-4-yl)cyclohexyl)butan-2-yl)benzo[d]thiazol-2-amine;
N-((R)-1-((cis)-4-(6-fluoroquinolin-4-yl)cyclohexyl)propan-2-yl)thiazolo[4,5-b]pyridin-2-amine;
N-((R)-1-((cis)-4-(6-fluoroquinolin-4-yl)cyclohexyl)propan-2-yl)-5-methoxythiazolo[5,4-b]pyridin-2-amine;
6-chloro-N-((R)-1-((cis)-4-(6-fluoroquinolin-4-yl)cyclohexyl)propan-2-yl)thiazolo[4,5-b]pyridin-2-amine;
N-((R)-1-((cis)-4-(6-fluoroquinolin-4-yl)cyclohexyl)propan-2-yl)benzo[d]oxazol-2-amine;
6-chloro-N-((R)-1-((cis)-4-(6-fluoroquinolin-4-yl)cyclohexyl)propan-2-yl)benzo[d]oxazol-2-amine;
N-((R)-1-((cis)-4-(6-fluoroquinolin-4-yl)cyclohexyl)propan-2-yl)-6-methoxybenzo[d]oxazol-2-amine;
6-chloro-N-((R)-1-((cis)-4-(6-fluoroquinolin-4-yl)cyclohexyl)butan-2-yl)benzo[d]oxazol-2-amine;
4-fluoro-N-((R)-1-((cis)-4-(6-fluoroquinolin-4-yl)cyclohexyl)propan-2-yl)benzo[d]oxazol-2-amine;
6-fluoro-N-((R)-1-((cis)-4-(6-fluoroquinolin-4-yl)cyclohexyl)propan-2-yl)benzo[d]oxazol-2-amine;
N-((R)-1-((cis)-4-(6-fluoroquinolin-4-yl)cyclohexyl)propan-2-yl)oxazolo[5,4-b]pyridin-2-amine;
6-chloro-N-((R)-1-((cis)-4-(6-fluoroquinolin-4-yl)cyclohexyl)propan-2-yl)oxazolo[4,5-b]pyridin-2-amine;
N-((R)-1-((cis)-4-(6-fluoroquinolin-4-yl)cyclohexyl)propan-2-yl)oxazolo[4,5-b]pyridin-2-amine;
6-fluoro-N-((R)-1-((cis)-4-(6-fluoroquinolin-4-yl)cyclohexyl)propan-2-yl)isoquinolin-1-amine;
6-chloro-N-((R)-1-((cis)-4-(6-fluoroquinolin-4-yl)cyclohexyl)propan-2-yl)isoquinolin-1-amine;
4-chloro-N-((R)-1-((cis)-4-(6-fluoroquinolin-4-yl)cyclohexyl)propan-2-yl)benzo[d]thiazol-2-amine;
5-fluoro-N-((R)-1-((cis)-4-(6-fluoroquinolin-4-yl)cyclohexyl)propan-2-yl)benzo[d]oxazol-2-amine;
7-fluoro-N-((R)-1-((cis)-4-(6-fluoroquinolin-4-yl)cyclohexyl)propan-2-yl)benzo[d]oxazol-2-amine;
2-chloro-N-((R)-1-((cis)-4-(6-fluoroquinolin-4-yl)cyclohexyl)propan-2-yl)quinazolin-4-amine;
6-chloro-N-((R)-1-((cis)-4-(6-fluoroquinolin-4-yl)cyclohexyl)propan-2-yl)phthalazin-1-amine;
2,7-dichloro-N-((R)-1-((cis)-4-(6-fluoroquinolin-4-yl)cyclohexyl)propan-2-yl)quinazolin-4-amine;
2,7-dichloro-N-((R)-1-((trans)-4-(6-fluoroquinolin-4-yl)cyclohexyl)propan-2-yl)quinazolin-4-amine;
7-fluoro-N-((R)-1-((cis)-4-(6-fluoroquinolin-4-yl)cyclohexyl)propan-2-yl)-6-methoxybenzo[d]oxazol-2-amine;
6-chloro-5-fluoro-N-((R)-1-((cis)-4-(6-fluoroquinolin-4-yl)cyclohexyl)propan-2-yl)benzo[d]oxazol-2-amine;
6-ethoxy-N-((R)-1-((cis)-4-(6-fluoroquinolin-4-yl)cyclohexyl)propan-2-yl)benzo[d]oxazol-2-amine;
6-(2-fluoroethoxy)-N-((R)-1-((cis)-4-(6-fluoroquinolin-4-yl)cyclohexyl)propan-2-yl)benzo[d]oxazol-2-amine;
6,7-dichloro-N-((R)-1-((cis)-4-(6-fluoroquinolin-4-yl)cyclohexyl)propan-2-yl)benzo[d]oxazol-2-amine;
6,7-difluoro-N-((R)-1-((cis)-4-(6-fluoroquinolin-4-yl)cyclohexyl)propan-2-yl)benzo[d]oxazol-2-amine;
2-chloro-6-fluoro-N-((R)-1-((trans)-4-(6-fluoroquinolin-4-yl)cyclohexyl)propan-2-yl)quinazolin-4-amine;
2,6-dichloro-N-((R)-1-((trans)-4-(6-fluoroquinolin-4-yl)cyclohexyl)propan-2-yl)quinazolin-4-amine;
2-chloro-8-fluoro-N-((R)-1-((trans)-4-(6-fluoroquinolin-4-yl)cyclohexyl)propan-2-yl)quinazolin-4-amine;
2,8-dichloro-N-((R)-1-((trans)-4-(6-fluoroquinolin-4-yl)cyclohexyl)propan-2-yl)quinazolin-4-amine;
2-chloro-N-((R)-1-((trans)-4-(6-fluoroquinolin-4-yl)cyclohexyl)propan-2-yl)-8-methoxyquinazolin-4-amine;
2-chloro-N-((R)-1-((trans)-4-(6-fluoroquinolin-4-yl)cyclohexyl)propan-2-yl)-8-(trifluoromethoxy)quinazolin-4-amine;
2-chloro-7-fluoro-N-((R)-1-((trans)-4-(6-fluoroquinolin-4-yl)cyclohexyl)propan-2-yl)quinazolin-4-amine;
2-chloro-4-(((R)-1-((trans)-4-(6-fluoroquinolin-4-yl)cyclohexyl)propan-2-yl)amino)quinazolin-7-carbonitrile;
7-bromo-2-chloro-N-((R)-1-((trans)-4-(6-fluoroquinolin-4-yl)cyclohexyl)propan-2-yl)quinazolin-4-amine;
2-chloro-N-((R)-1-((trans)-4-(6-fluoroquinolin-4-yl)cyclohexyl)propan-2-yl)-7-methylquinazolin-4-amine;
2-chloro-N-((R)-1-((trans)-4-(6-fluoroquinolin-4-yl)cyclohexyl)propan-2-yl)-7-(trifluoromethyl)quinazolin-4-amine;
2-chloro-N-((R)-1-((trans)-4-(6-fluoroquinolin-4-yl)cyclohexyl)propan-2-yl)-7-methoxyquinazolin-4-amine;
2-chloro-6-fluoro-N-((R)-1-((cis)-4-(6-fluoroquinolin-4-yl)cyclohexyl)propan-2-yl)quinazolin-4-amine;
2,6-dichloro-N-((R)-1-((cis)-4-(6-fluoroquinolin-4-yl)cyclohexyl)propan-2-yl)quinazolin-4-amine;
2-chloro-7-fluoro-N-((R)-1-((cis)-4-(6-fluoroquinolin-4-yl)cyclohexyl)propan-2-yl)quinazolin-4-amine;
2-chloro-4-(((R)-1-((cis)-4-(6-fluoroquinolin-4-yl)cyclohexyl)propan-2-yl)amino)quinazolin-7-carbonitrile;
7-bromo-2-chloro-N-((R)-1-((cis)-4-(6-fluoroquinolin-4-yl)cyclohexyl)propan-2-yl)quinazolin-4-amine;
2-chloro-N-((R)-1-((cis)-4-(6-fluoroquinolin-4-yl)cyclohexyl)propan-2-yl)-7-methylquinazolin-4-amine;
2-chloro-N-((R)-1-((cis)-4-(6-fluoroquinolin-4-yl)cyclohexyl)propan-2-yl)-7-(trifluoromethyl)quinazolin-4-amine;
2-chloro-N-((R)-1-((cis)-4-(6-fluoroquinolin-4-yl)cyclohexyl)propan-2-yl)-7-methoxyquinazolin-4-amine;
2,8-dichloro-N-((R)-1-((cis)-4-(6-fluoroquinolin-4-yl)cyclohexyl)propan-2-yl)quinazolin-4-amine;
2-chloro-8-fluoro-N-((R)-1-((cis)-4-(6-fluoroquinolin-4-yl)cyclohexyl)propan-2-yl)quinazolin-4-amine;
2-chloro-N-((R)-1-((cis)-4-(6-fluoroquinolin-4-yl)cyclohexyl)propan-2-yl)-8-methoxyquinazolin-4-amine; and
2-chloro-N-((R)-1-((cis)-4-(6-fluoroquinolin-4-yl)cyclohexyl)propan-2-yl)-8-(trifluoromethoxy)quinazolin-4-amine.

12. The compound or pharmaceutically acceptable salt thereof as claimed in claim 1, which is selected from the group consisting of:
7-bromo-N-((R)-1-((cis)-4-(6-fluoroquinolin-4-yl)cyclohexyl)propan-2-yl)quinazolin-4-amine;
7-bromo-4-(((R)-1-((trans)-4-(6-fluoroquinolin-4-yl)cyclohexyl)propan-2-yl)amino)-2H-chromen-2-one;
6-chloro-N-((R)-1-((cis)-4-(6-fluoroquinolin-4-yl)cyclohexyl)propan-2-yl)benzo[d]oxazol-2-amine;
6-chloro-N-((R)-1-((cis)-4-(6-fluoroquinolin-4-yl)cyclohexyl)butan-2-yl)benzo[d]oxazol-2-amine;
4-fluoro-N-((R)-1-((cis)-4-(6-fluoroquinolin-4-yl)cyclohexyl)propan-2-yl)benzo[d]oxazol-2-amine;
6-fluoro-N-((R)-1-((cis)-4-(6-fluoroquinolin-4-yl)cyclohexyl)propan-2-yl)benzo[d]oxazol-2-amine; and
6,7-difluoro-N-((R)-1-((cis)-4-(6-fluoroquinolin-4-yl)cyclohexyl)propan-2-yl)benzo[d]oxazol-2-amine.

13. A process for preparing a compound of Formula 1 or pharmaceutically acceptable salt thereof as claimed in claim 1, the process of which comprises reacting a compound of Formula 2 or salt thereof with a compound of Formula 3 to obtain a compound of Formula 1; and optionally converting the compound of Formula 1 to a pharmaceutically acceptable salt thereof: 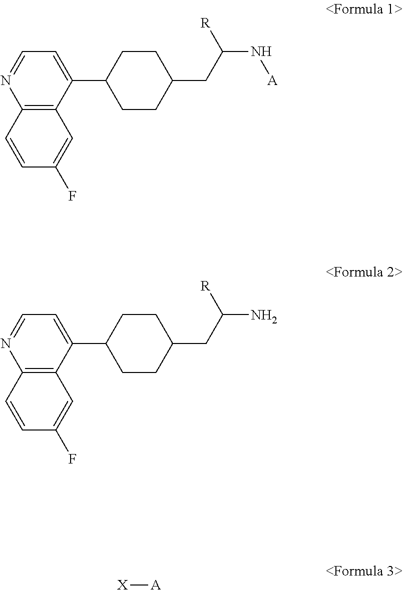
wherein, R and A are the same as defined in claim 1; and X is halogen.

14. A compound of Formula 2 or salt thereof: 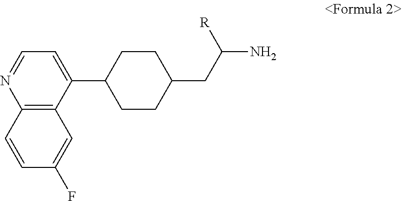
wherein, R is a C₁-C₆ alkyl group or a C₃-C₁₀ cycloalkyl group.

15. A pharmaceutical composition for inhibiting indoleamine 2,3-dioxygenase comprising the compound or pharmaceutically acceptable salt thereof as claimed in claim 1 as an active ingredient.

16. The pharmaceutical composition as claimed in claim 15 for preventing or treating viral infection; rheumatoid arthritis; cancer selected from the group consisting of melanoma, pancreatic cancer, prostate cancer, and brain cancer; or depression.

17. A method for inhibiting indoleamine 2,3-dioxygenase in a mammal, which comprises administering a therapeutically effective amount of the compound or pharmaceutically acceptable salt thereof as claimed in claim 1 to the mammal in need thereof.
